(19) United States
(12) Patent Application Publication
(10) Pub. No.: US 2011/0028479 A1
(43) Pub. Date: Feb. 3, 2011

(54) SUBSTITUTED PYRROLOTRIAZINES AND THEIR USE

(75) Inventors: Thomas Lampe, Dusseldorf (DE); Raimund Kast, Wuppertal (DE); Friederike Stoll, Dusseldorf (DE); Joachim Schuhmacher, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT

(21) Appl. No.: 12/740,680

(22) PCT Filed: Oct. 16, 2008
(86) PCT No.: PCT/EP08/08764

§ 371 Date: Oct. 8, 2010

(30) Foreign Application Priority Data
Oct. 30, 2007 (DE) 10 2007 051 762.0

(51) Int. Cl. A61K 31/53; C07D 253/10; A61P 9/00

(52) U.S. Cl. 514/243; 544/183

(57) ABSTRACT

The present application relates to new, substituted pyrrolo[2,1-f][1,2,4]triazine derivatives of the formula (I) as prostacyclin (PGI₂) IP receptor activators for the treatment and/or prophylaxis of cardiovascular disorders.

The present application relates to novel substituted pyrrolo[2,1-f][1,2,4]triazine derivatives, to processes for their preparation, to their use for the treatment and/or prophylaxis of diseases and to their use for preparing medicaments for the treatment and/or prophylaxis of diseases, in particular for the treatment and/or prophylaxis of cardiovascular diseases.

Prostacyclin (PGI₂) belongs to the class of bioactive prostaglandins, which are derivatives of arachidonic acid. PGI₂ is the main product of arachidonic acid metabolism in endothelial cells and is a potent vasodilator and inhibitor of platelet aggregation. PGI₂ is the physiological antagonist of thromboxane A₂ (TxA₂), a strong vasoconstrictor and stimulator of thrombocyte aggregation, and thus contributes to the maintenance of vascular homeostasis. A drop in PGI₂ levels is presumed to be partly responsible for the development of various cardiovascular diseases [Dusting, G. J. et al., Pharmac. Ther. 1990, 48: 323-344; Vane, J. et al., Eur. J. Vasc. Endovasc. Surg. 2003, 26: 571-578].

After release of arachidonic acid from phospholipids via phospholipases A₂, PGI₂ is synthesized by cyclooxygenases and then by PGI₂-synthase. PGI₂ is not stored, but is released immediately after synthesis, exerting its effects locally. PGI₂ is an unstable molecule, which is transformed rapidly (half-life approx. 3 minutes) and non-enzymatically, to an inactive metabolite, 6-keto-prostaglandin+1 alpha [Dusting, G. J. et al., Pharmac. Ther. 1990, 48: 323-344].

The biological effects of PGI₂ occur through binding to a membrane-bound receptor, called the prostacyclin receptor or IP receptor [Narumiya, S. et al., Physiol. Rev. 1999, 79: 1193-1226]. The IP receptor is one of the G-protein-coupled receptors, which are characterized by seven transmembrane domains. In addition to the human IP receptor, prostacyclin receptors have also been cloned from rat and mouse [Vane, J. et al., Eur. J. Vasc. Endovasc. Surg. 2003, 26: 571-578]. In smooth muscle cells, activation of the IP receptor leads to stimulation of adenylate cyclase, which catalyses the formation of cAMP from ATP. The increase in the intracellular cAMP concentration is responsible for prostacyclin-induced vasodilation and for inhibition of platelet aggregation. In addition to the vasoactive properties, anti-proliferative effects [Schroer, K. et al., Agents Actions Suppl. 1997, 48: 63-91; Kothapalli, D. et al., Mol. Pharmacol. 2003, 64: 249-258; Planchon, P. et al., Life Sci. 1995, 57: 1233-1240] and anti-arteriosclerotic effects [Rudic, R. D. et al., Circ. Res. 2005, 96: 1240-1247; Egan K. M. et al., Science 2004, 114: 784-794] have also been described for PGI₂. Furthermore, PGI₂ also inhibits the formation of metastases [Schneider, M. R. et al., Cancer Metastasis Rev. 1994, 13: 349-64]. It is unclear whether these effects are due to stimulation of cAMP formation or to IP receptor-mediated activation of other signal transduction pathways in the respective target cell [Wise, H. et al. TIPS 1996, 17: 17-21], such as the phosphoinositide cascade, and of potassium channels.

Although the effects of PGI₂ are on the whole of benefit therapeutically, clinical application of PGI₂ is severely restricted by its chemical and metabolic instability. PGI₂ analogs that are more stable, for example iloprost [Badesch, D. B. et al., J. Am. Coll. Cardiol. 2004, 43: 56S-61S] and treprostinil [Chattaraj, S. C., Curr. Opion. Invest. Drugs 2002, 3: 582-586] have been made available, but these compounds still have a very short time of action. Moreover, the substances can only be administered to the patient via complicated routes of administration, e.g. by continuous infusion, subcutaneously or via repeated inhalations. These routes of administration can also have additional side-effects, for example infections or pains at the site of injection. The use of beraprost, which to date is the only PGI₂ derivative available for oral administration to the patient [Barst, R. J. et al., J. Am. Coll. Cardiol. 2003, 41: 2119-2125], is once again limited by its short time of action.

It is the object of the present invention to provide novel substances which act as chemically and metabolically stable, orally available activators of the IP receptor and, as such, are suitable for treating diseases, in particular cardiovascular diseases. WO 03/018589 describes 4-aminofuro[2,3-d]pyrimidines as adenosinkinase inhibitors for treating cardiovascular diseases. Other 4-amino-, 4-oxy- or 4-thio-substituted furo[2,3-d]pyrimidine derivatives and their use for treating cardiovascular diseases are described in WO 2007/079861 and WO 2007/079862. Compounds having a pyrrolo[2,1-f][1,2,4]triazine partial structure are described in WO 2006/004833 as protein kinase inhibitors for treating neoplastic diseases. WO 2006/004884 claims processes for preparing certain pyrrolo[2,1-f][1,2,4]triazine derivatives.

The present invention provides compounds of the general formula (I)

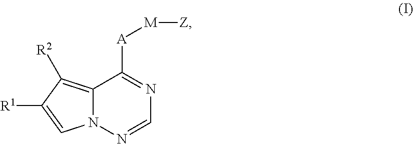

in which
A represents O or N—R³ in which
R³ represents hydrogen, (C₁-C₆)-alkyl, (C₃-C₇)-cycloalkyl or (C₄-C₇)-cycloalkenyl,
M represents a group of the formula

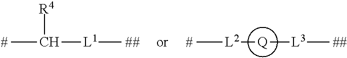

in which
# represents the point of attachment to the group A
and
## represents the point of attachment to the group Z,
R⁴ represents hydrogen or (C₁-C₄)-alkyl which may be substituted by hydroxyl or amino,
L¹ represents (C₁-C₇)-alkanediyl or (C₂-C₇)-alkenediyl which may be mono- or disubstituted by fluorine, or a group of the formula *-L^{1A}-V-L^{1B}-** in which
* represents the point of attachment to the group —CHR⁴,
** represents the point of attachment to the group Z,
L^{1A} represents (C₁-C₅)-alkanediyl which may be mono- or disubstituted by identical or different radicals from the group consisting of (C₁-C₄)-alkyl and (C₁-C₄)-alkoxy,
L^{1B} represents a bond or (C₁-C₃)-alkanediyl which may be mono- or disubstituted by fluorine,
and
V O or N—R⁵ in which
R⁵ represents hydrogen, (C₁-C₆)-alkyl or (C₃-C₇)-cycloalkyl,
L² represents a bond or (C₁-C₄)-alkanediyl,
L³ represents (C₁-C₄)-alkanediyl which may be mono- or disubstituted by fluorine and in which a methylene group may be replaced by O or N—R⁶ in which
R⁶ represents hydrogen, (C₁-C₆)-alkyl or (C₃-C₇)-cycloalkyl,
or represents (C₂-C₄)-alkenediyl,
and
Q represents (C₃-C₇)-cycloalkyl, (C₄-C₇)-cycloalkenyl, phenyl, 5- to 7-membered heterocyclyl or 5- or 6-membered heteroaryl, each of which may be substituted up to two times by identical or different radicals from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino,
where (C₁-C₄)-alkyl for its part may be substituted by hydroxyl, (C₁-C₄)-alkoxy, amino, mono- or di-(C₁-C₄)-alkylamino,
Z represents a group of the formula

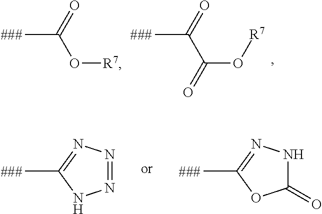

in which
### represents the point of attachment to the group L¹ or L³
and
R⁷ represents hydrogen or (C₁-C₄)-alkyl,
and
R¹ and R² are identical or different and independently of one another represent (C₃-C₇)-cycloalkyl, (C₄-C₇)-cycloalkenyl, phenyl, 5- to 7-membered heterocyclyl or 5- or 6-membered heteroaryl, each of which may be mono- to trisubstituted by identical or different radicals from the group consisting of halogen, cyano, nitro, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-acyl, amino, mono-(C₁-C₆)-alkylamino, di-(C₁-C₆)-alkylamino and (C₁-C₆)-acylamino,
where (C₁-C₆)-alkyl and (C₁-C₆)-alkoxy for their part may in each case be substituted by cyano, hydroxyl, (C₁-C₄)-alkoxy, (C₁-C₄)-alkylthio, amino, mono- or di-(C₁-C₄)-alkylamino,
or
R¹ and/or R² represent(s) phenyl in which two radicals attached to adjacent ring carbon atoms together form a group of the formula —O—CH₂—O—, —O—CHF—O—, —O—CF₂—O—, —O—CH₂—CH₂—O— or —O—CF₂—CF₂—O—,
and their salts, solvates and solvates of the salts.

Compounds according to the invention are the compounds of the formula (I) and the salts, solvates and solvates of the salts thereof, the compounds of the formulae below encompassed by the formula (I) and the salts, solvates and solvates of the salts thereof, and also the compounds encompassed by the formula (I) and mentioned below as working examples, and the salts, solvates and solvates of the salts thereof, provided the compounds encompassed by formula (I) and mentioned below are not already salts, solvates and solvates of the salts.

The compounds of the invention may, depending on their structure, exist in stereoisomeric forms (enantiomers, diastereomers). The present invention therefore relates to the enantiomers or diastereomers and respective mixtures thereof. The stereoisomerically pure constituents can be isolated in a known manner from such mixtures of enantiomers and/or diastereomers.

If the compounds of the invention may occur in tautomeric forms, the present invention encompasses all tautomeric forms.

Salts which are preferred for the purposes of the present invention are physiologically acceptable salts of the compounds of the invention. Also encompassed are salts which are themselves unsuitable for pharmaceutical uses but can be used for example for isolating or purifying the compounds of the invention. Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid. Physiologically acceptable salts of the compounds of the invention also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refers for the purposes of the invention to those forms of the compounds of the invention which form, in the solid or liquid state, a complex by coordination with solvent molecules. Hydrates are a specific form of solvates in which the coordination takes place with water. Hydrates are preferred solvates in the context of the present invention.

The present invention additionally encompasses prodrugs of the compounds of the invention. The term “prodrugs” encompasses compounds which themselves may be biologically active or inactive, but are converted during their residence time in the body into compounds of the invention (for example by metabolism or hydrolysis).

In particular, for the compounds of the formula (I) in which

Z represents a group of the formula

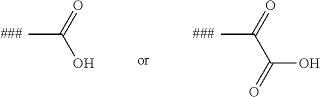

the present invention also includes hydrolyzable ester derivatives of these compounds. These are to be understood as meaning esters which can be hydrolyzed to the free carboxylic acids, as the compounds that are mainly active biologically, in physiological media, under the conditions of the biological tests described later and in particular in vivo by enzymatic or chemical routes. (C₁-C₄)-alkyl esters, in which the alkyl group can be straight-chain or branched, are preferred as such esters. Particular preference is given to methyl or ethyl esters (see also the corresponding definitions of the radical R⁷).

In the context of the present invention, the substituents have the following meaning, unless specified otherwise:

(C₁-C₆)-Alkyl, (C₁-C₆)-alkyl, (C₁-C₄)-alkyl and (C₁-C₃)-alkyl stand in the context of the invention for a straight-chain or branched alkyl radical having respectively 1 to 6, 1 to 5, 1 to 4 and 1 to 3 carbon atoms. A straight-chain or branched alkyl radical having 1 to 4, in particular 1 to 3, carbon atoms is preferred. Examples which may be preferably mentioned are: methyl, ethyl, n-propyl, isopropyl, n-butyl, isobutyl, sec-butyl, tert-butyl, 1-ethylpropyl, n-pentyl and n-hexyl.

(C₂-C₆)-Alkenyl, (C₂-C₅)-alkenyl and (C₂-C₄)-alkenyl stand in the context of the invention for a straight-chain or branched alkenyl radical having respectively 2 to 6, 2 to 5 and 2 to 4 carbon atoms and one or two double bonds. A straight-chain or branched alkenyl radical having 2 to 4 carbon atoms and one double bond is preferred. Examples which may be preferably mentioned are: vinyl, allyl, isopropenyl and n-but-2-en-1-yl.

(C₂-C₄)-Alkynyl stands in the context of the invention for a straight-chain or branched alkynyl radical having 2 to 4 carbon atoms and one triple bond. A straight-chain alkynyl radical having 2 to 4 carbon atoms is preferred. Examples which may be preferably mentioned are: ethynyl, n-prop-1-in-1-yl, n-prop-2-in-1-yl, n-but-2-in-1-yl and n-but-3-in-1-yl.

(C₁-C₄)-Alkanediyl and (C₁-C₃)-alkanediyl stand in the context of the invention for a straight-chain or branched divalent alkyl radical having respectively 1 to 4 and 1 to 3 carbon atoms. In each case, a straight-chain alkanediyl radical having respectively 1 to 4 and 1 to 3 carbon atoms is preferred. Examples which may be preferably mentioned are: methylene, ethane-1,2-diyl (1,2-ethylene), ethane-1,1-diyl, propane-1,3-diyl (1,3-propylene), propane-1,1-diyl, propane-1,2-diyl, propane-2,2-diyl, butane-1,4-diyl (1,4-butylene), butane-1,2-diyl, butane-1,3-diyl and butane-2,3-diyl.

(C₁-C₇)-Alkanediyl, (C₁-C₅)-alkanediyl and (C₃-C₇)-alkanediyl stand in the context of the invention for a straight-chain or branched divalent alkyl radical having respectively 1 to 7, 1 to 5 and 3 to 7 carbon atoms. In each case, a straight-chain alkanediyl radical having respectively 1 to 7, 1 to 5 and 3 to 7 carbon atoms is preferred. Examples which may be preferably mentioned are: methylene, ethane-1,2-diyl (1,2-ethylene), ethane-1,1-diyl, propane-1,3-diyl (1,3-propylene), propane-1,1-diyl, propane-1,2-diyl, propane-2,2-diyl, butane-1,4-diyl (1,4-butylene), butane-1,2-diyl, butane-1,3-diyl, butane-2,3-diyl, pentane-1,5-diyl (1,5-pentylene), pentane-2,4-diyl, 3-methylpentane-2,4-diyl and hexane-1,6-diyl(1,6-hexylene).

(C₂-C₄)-Alkenediyl and (C₂-C₃)-alkenediyl stand in the context of the invention for a straight-chain or branched divalent alkenyl radical having respectively 2 to 4 and 2 to 3 carbon atoms and up to 2 double bonds. In each case, a straight-chain alkenediyl radical having respectively 2 to 4 and 2 to 3 carbon atoms and one double bond is preferred. Examples which may be preferably mentioned are: ethene-1,1-diyl, ethene-1,2-diyl, propene-1,1-diyl, propene-1,2-diyl, propene-1,3-diyl, but-1-ene-1,4-diyl, but-1-ene-1,3-diyl, but-2-ene-1,4-diyl and buta-1,3-diene-1,4-diyl.

(C₂-C₇)-Alkenediyl and (C₃-C₇)-alkenediyl stand in the context of the invention for a straight-chain or branched divalent alkenyl radical having respectively 2 to 7 and 3 to 7 carbon atoms and up to 3 double bonds. In each case, a straight-chain alkenediyl radical having respectively 2 to 7 and 3 to 7 carbon atoms and one double bond is preferred. Examples which may be preferably mentioned are: ethene-1,1-diyl, ethene-1,2-diyl, propene-1,1-diyl, propene-1,2-diyl, propene-1,3-diyl, but-1-ene-1,4-diyl, but-1-ene-1,3-diyl, but-2-ene-1,4-diyl, buta-1,3-diene-1,4-diyl, pent-2-ene-1,5-diyl, hex-3-ene-1,6-diyl and hexa-2,4-diene-1,6-diyl.

(C₁-C₆)-Alkoxy and (C₁-C₄)-alkoxy stand in the context of the invention for a straight-chain or branched alkoxy radical having respectively 1 to 6 and 1 to 4 carbon atoms. A straight-chain or branched alkoxy radical having 1 to 4 carbon atoms is preferred. Examples which may be preferably mentioned are: methoxy, ethoxy, n-propoxy, isopropoxy, n-butoxy, tert-butoxy, n-pentoxy and n-hexoxy.

(C₁-C₆)-Alkylthio and (C₁-C₄)-alkylthio stand in the context of the invention for a straight-chain or branched alkylthio radical having respectively 1 to 6 and 1 to 4 carbon atoms. A straight-chain or branched alkylthio radical having 1 to 4 carbon atoms is preferred. Examples which may be preferably mentioned are: methylthio, ethylthio, n-propylthio, isopropylthio, n-butylthio, tert-butylthio, n-pentylthio and n-hexylthio.

(C₁-C₆)-Acyl [(C₁-C₆)-alkanoyl], (C₁-C₅)-acyl [(C₁-C₅)-alkanoyl] and (C₁-C₄)-acyl [(C₁-C₄)-alkanoyl] stand in the context of the invention for a straight-chain or branched alkyl radical having respectively 1 to 6, 1 to 5 and 1 to 4 carbon atoms which carries a doubly attached oxygen atom in the 1-position and is attached via the 1-position. A straight-chain or branched acyl radical having 1 to 4 carbon atoms is preferred. Examples which may be preferably mentioned are: formyl, acetyl, propionyl, n-butyryl, isobutyryl and pivaloyl.

Mono-(C₁-C₆)-alkylamino and mono-(C₁-C₄)-alkylamino stand in the context of the invention for an amino group having a straight-chain or branched alkyl substituent which has respectively 1 to 6 and 1 to 4 carbon atoms. A straight-chain or branched monoalkylamino radical having 1 to 4 carbon atoms is preferred. Examples which may be preferably mentioned are: methylamino, ethylamino, n-propylamino, isopropylamino and tert-butylamino.

Di-(C₁-C₆)-alkylamino and di-(C₁-C₄)-alkylamino stand in the context of the invention for an amino group having two identical or different straight-chain or branched alkyl substituents having respectively 1 to 6 and 1 to 4 carbon atoms. Straight-chain or branched dialkylamino radicals having in each case 1 to 4 carbon atoms are preferred. Examples which may be preferably mentioned are: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

(C₁-C₆)-Acylamino and (C₁-C₄)-acylamino stand in the context of the invention for an amino group having a straight-chain or branched acyl substituent which has respectively 1 to 6 and 1 to 4 carbon atoms and is attached via the carbonyl group. An acylamino radical having 1 to 4 carbon atoms is preferred. Examples which may be preferably mentioned are: formamido, acetamido, propionamido, n-butyramido and pivaloylamido.

(C₃-C₇)-Cycloalkyl, (C₃-C₆)-cycloalkyl and (C₄-C₆)-cycloalkyl stand in the context of the invention for a monocyclic saturated cycloalkyl group having respectively 3 to 7, 3 to 6 and 4 to 6 carbon atoms. A cycloalkyl radical having 3 to 6 carbon atoms is preferred. Examples which may be preferably mentioned are: cyclopropyl, cyclobutyl, cyclopentyl, cyclohexyl and cycloheptyl.

(C₄-C₇)-Cycloalkenyl, (C₄-C₆)-cycloalkenyl and (C₅-C₆)-cycloalkenyl stand in the context of the invention for a monocyclic cycloalkyl group having respectively 4 to 7, 4 to 6 and 5 or 6 carbon atoms and one double bond. A cycloalkenyl radical having 4 to 6, particularly preferably 5 or 6, carbon atoms is preferred. Examples which may be preferably mentioned are: cyclobutenyl, cyclopentenyl, cyclohexenyl and cycloheptenyl.

5- to 7-membered heterocyclyl stands in the context of the invention for a saturated or partially unsaturated heterocycle having a total of 5 to 7 ring atoms which contains one or two ring heteratoms from the group consisting of N and O and is attached via ring carbon atoms and/or, if appropriate, ring nitrogen atoms. A 5- or 6-membered saturated heterocycle having one or two ring heteroatoms from the group consisting of N and O is preferred. Examples which may be mentioned are: pyrrolidinyl, pyrrolinyl, pyrazolidinyl, tetrahydrofuranyl, piperidinyl, piperazinyl, dihydropyranyl, tetrahydropyranyl, morpholinyl, hexahydroazepinyl and hexahydro-1,4-diazepinyl. Preference is given to pyrrolidinyl, tetrahydrofuranyl, piperidinyl, piperazinyl, tetrahydropyranyl and morpholinyl.

5- or 6-membered heteroaryl stands in the context of the invention for an aromatic heterocycle (heteroaromatic) having a total of 5 or 6 ring atoms which contains one or two ring heteroatoms from the group consisting of N, O and S and is attached via ring carbon atoms and/or, if appropriate, a ring nitrogen atom. Examples which may be mentioned are: furyl, pyrrolyl, thienyl, pyrazolyl, imidazolyl, thiazolyl, oxazolyl, isoxazolyl, isothiazolyl, pyridyl, pyrimidinyl, pyridazinyl and pyrazinyl. Preference is given to thienyl, pyridyl, pyrimidinyl, pyridazinyl and pyrazinyl.

Halogen includes in the context of the invention fluorine, chlorine, bromine and iodine. Preference is given to chlorine or fluorine.

If radicals in the compounds according to the invention are substituted, the radicals, unless specified otherwise, may be mono- or polysubstituted. In the context of the present invention, for all radicals that occur more than once, their meanings are independent of one another. Substitution by one, two or three identical or different substituents is preferred. Particular preference is given to substitution with one or two identical or different substituents, very particular preference is given to substitution by one substituent.

In the context of the present invention, preference is given to compounds of the formula (I) in which

A represents O or NH,
M represents a group of the formula

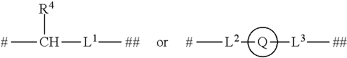

in which
# represents the point of attachment to the group A
and
## represents the point of attachment to the group Z,
R⁴ represents hydrogen, methyl or ethyl,
L¹ represents (C₃-C₇)-alkanediyl, (C₃-C₇)-alkenediyl or a group of the formula *-L^{1A}-V-L^{1B}-** in which
* represents the point of attachment to the group —CHR⁴,
** represents the point of attachment to the group Z,
L^{1A} represents (C₁-C₃)-alkanediyl which may be mono- or disubstituted by methyl,
L^{1B} represents (C₁-C₃)-alkanediyl
and
V represents O or N—CH₃,
L² represents a bond, methylene, ethane-1,1-diyl or ethane-1,2-diyl,
L³ represents (C₁-C₃)-alkanediyl or a group of the formula -W—CH₂- or
-W—CH₂—CH₂- in which
 represents the point of attachment to the ring Q,
 represents the point of attachment to the group Z
and
W represents O or N—R⁶, in which
R⁶ represents hydrogen or (C₁-C₃)-alkyl,
and
Q represents cyclobutyl, cyclopentyl, cyclopentenyl, cyclohexyl, pyrrolidinyl, piperidinyl, tetrahydrofuranyl, tetrahydropyranyl, morpholinyl or phenyl, each of which may be substituted up to two times by identical or different radicals from the group consisting of fluorine, methyl, ethyl, trifluoromethyl, hydroxyl, methoxy and ethoxy,
Z represents a group of the formula

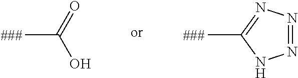

in which
### represents the point of attachment to the group L¹ or L³,
and
R¹ and R² are identical or different and independently of one another represent cyclopenten-1-yl, cyclohexen-1-yl, phenyl, thienyl or pyridyl, each of which may be mono- or disubstituted by identical or different radicals from the group consisting of fluorine, chlorine, cyano, (C₁-C₄)-alkyl, (C₂-C₄)-alkenyl, (C₁-C₄)-alkoxy, trifluoromethyl and trifluoromethoxy,
and to their salts, solvates and solvates of the salts.

In the context of the present invention, particular preference is given to compounds of the formula (I) in which

A represents O or NH,
M represents the group of the formula

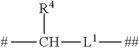

in which
# represents the point of attachment to the group A
and
## represents the point of attachment to the group Z,
R⁴ represents hydrogen or methyl,
and
L¹ represents butane-1,4-diyl, pentane-1,5-diyl or a group of the formula *L^{1A}-O-L^{1B}-** in which
* represents the point of attachment to the group —CHR⁴,
** represents the point of attachment to the group Z,
L^{1A} represents methylene or ethane-1,2-diyl which may be mono- or disubstituted by methyl,
and
L^{1B} represents methylene or ethane-1,2-diyl,
Z represents the group of the formula

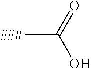

in which
### represents the point of attachment to the group L¹,
R¹ represents phenyl which may be substituted by fluorine or chlorine,
and
R² represents phenyl which may be substituted by methyl, ethyl, methoxy or ethoxy,
and to their salts, solvates and solvates of the salts.

The individual definitions of radicals given in the respective combinations and preferred combinations of radicals are, independently of the given combination of radicals in question, also replaced by radical definitions of other combinations. Particular preference is given to combinations of two or more of the preferred ranges mentioned above.

In the context of the present invention, very particular preference is given to the compounds mentioned below:
(6-{[5-(4-methoxyphenyl)-6-phenylpyrrolo[2,1-f][1,2,4]triazin-4-yl]amino}hexanoic acid
   and
(6R)-6-{[5-(4-methoxyphenyl)-6-phenylpyrrolo[2,1-f][1,2,4]triazin-4-yl]oxy}heptanoic acid
   and to their salts, solvates and solvates of the salts.

The invention furthermore provides a process for preparing the compounds of the formula (I) according to the invention in which Z represents —COOH or —C(═O)—COON, characterized in that either

[A] compounds of the formula (II)

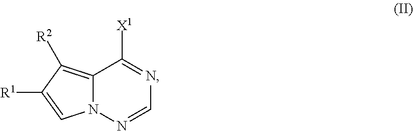

in which R¹ and R² have the meanings given above
and
X¹ represents a leaving group such as, for example, halogen, in particular chlorine,
are reacted in an inert solvent in the presence of a base with a compound of the formula (III)

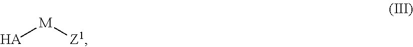

in which A and M have the meanings given above
and
Z¹ represents cyano or a group of the formula —[C(O)]_{y}—COOR^{7A} in which
y represents the number 0 or 1
and
R^{7A} represents (C₁-C₄)-alkyl,
to give compounds of the formula (IV)

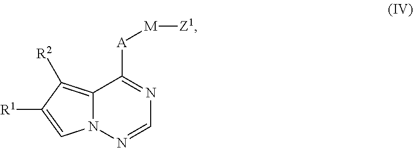

in which A, M, Z¹, R¹ and R² each have the meanings given above,
or
[B] compounds of the formula (V)

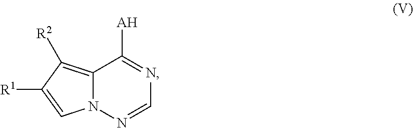

in which A, R¹ and R² each have the meanings given above,
are reacted in an inert solvent in the presence of a base with a compound of the formula (VI)

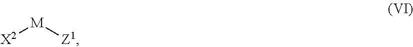

in which M and Z¹ have the meanings given above
and
X² represents a leaving group such as, for example, halogen, mesylate, tosylate or triflate,
to give compounds of the formula (IV)

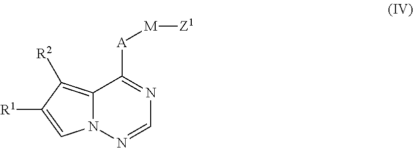

in which A, M, Z¹, R¹ and R² each have the meanings given above,
and the compounds of the formula (IV) are then converted by hydrolysis of the ester or cyano group Z¹ into the carboxylic acids of the formula (I-A)

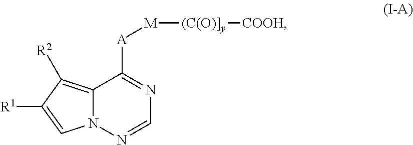

in which A, M, R¹, R² and y each have the meanings given above,
and these are, if appropriate, converted into their solvates, salts and/or solvates of the salts using the appropriate (i) solvents and/or (ii) bases or acids.

Inert solvents for process steps (II)+(III)→(IV) and (V)+(VI)→(IV) are, for example, ethers, such as diethyl ether, methyl tert-butyl ether, dioxane, tetrahydro-furan, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethane, tetrachloroethane, trichloro-ethylene, chlorobenzene or chlorotoluene, or other solvents, such as dimethyl-formamide (DMF), dimethyl sulfoxide (DMSO), N,N′-dimethylpropyleneurea (DMPU), N-methylpyrrolidone (NMP) or acetonitrile. It is also possible to use mixtures of the solvents mentioned. Preference is given to using tetrahydrofuran, toluene, dimethylformamide, dimethyl sulfoxide or mixtures of these solvents.

However, if appropriate, the process steps (II)+(III)→(IV) and (V)+(VI) →(IV) can also be carried out in the absence of a solvent.

Suitable bases for the process steps (II)+(III)→(IV) and (V)+(VI)→(IV) are customary inorganic or organic bases. These preferably include alkali metal hydroxides, such as, for example, lithium hydroxide, sodium hydroxide, or potassium hydroxide, alkali metal or alkaline earth metal carbonates, such as lithium carbonate, sodium carbonate, potassium carbonate, calcium carbonate or cesium carbonate, alkali metal alkoxides, such as sodium tert-butoxide or potassium tert-butoxide, alkali metal hydrides, such as sodium hydride or potassium hydride, amides, such as lithium bis(trimethylsilyl)amide or potassium bis(trimethylsilyl)amide or lithium diisopropylamide, organic metallic compounds, such as butyllithium or phenyllithium, or organic amines, such as triethylamine, N-methylmorpholine, N-methylpiperidine, N,N-diisopropylethylamine or pyridine.

In the case of the reaction with alcohol derivatives [A in (III) and (V)=O], phosphazene bases (so-called “Schwesinger bases”), such as, for example, P2-t-Bu or P4-t-Bu are likewise expedient [cf., for example, R. Schwesinger, H. Schlemper, Angew. Chem. Int. Ed. Engl. 26, 1167 (1987); T. Pietzonka, D. Seebach, Chem. Ber. 124, 1837 (1991)].

In the reaction with amine derivatives [A in (III) and (V)=N], the base used is preferably a tertiary amine, such as, in particular, N,N-diisopropylethylamine, sodium tert-butoxide or sodium hydride. However, if appropriate, these reactions can—if an excess of the amine component (III) is used—also be carried out without the addition of an auxiliary base. In the reaction with alcohol derivatives [A in (III) and (V)=O], preference is given to sodium hydride, potassium carbonate or cesium carbonate or the phosphazene bases P2-t-Bu and P4-t-Bu.

If appropriate, the process steps (II)+(III)→(IV) and (V)+(VI)→(IV) can advantageously be carried out with addition of a crown ether.

In one process variant, the reactions (II)+(III)→(IV) and (V)+(VI)→(IV) can also be carried out in a two-phase mixture consisting of an aqueous alkali metal hydroxide solution as base and one of the hydrocarbons or halogenated hydrocarbons mentioned above as further solvent, using a phase-transfer catalyst, such as tetrabutylammonium hydrogen sulfate or tetrabutylammonium bromide.

The process steps (II)+(III)→(IV) and (V)+(VI)→(IV) are, in the reaction with amine derivatives [A in (III) and (V)=N], generally carried out in a temperature range of from −20° C. to +150° C., preferably at from 0° C. to +100° C. In the reaction with alcohol derivatives [A in (III) and (V)=O], the reactions are generally carried out in a temperature range of from −20° C. to +120° C., preferably at from −10° C. to +80° C.

The hydrolysis of the ester or nitrile group Z¹ in process step (IV)→(I-A) is carried out by customary methods by treating the esters or nitriles in inert solvents with acids or bases, where in the latter case the salts initially formed are converted by treatment with acid into the free carboxylic acids. In the case of the tert-butyl esters, the ester cleavage is preferably carried out using acids.

Suitable inert solvents for these reactions are water or the organic solvents customary for ester cleavage. These preferably include alcohols, such as methanol, ethanol, n-propanol, isopropanol, n-butanol or tert-butanol, or ethers, such as diethyl ether, tetrahydrofuran, dioxane or glycol dimethyl ether, or other solvents, such as acetone, dichloromethane, dimethylformamide or dimethyl sulfoxide. It is also possible to use mixtures of the solvents mentioned. In the case of a basic ester hydrolysis, preference is given to using mixtures of water with dioxane, tetrahydrofuran, methanol and/or ethanol, and for nitrile hydrolysis, preference is given to using water and/or n-propanol. In the case of the reaction with trifluoroacetic acid, preference is given to using dichloromethane, and in the case of the reaction with hydrogen chloride, preference is given to using tetrahydrofuran, diethyl ether, dioxane or water.

Suitable bases are the customary inorganic bases. These preferably include alkali metal hydroxides or alkaline earth metal hydroxides, such as, for example, sodium hydroxide, lithium hydroxide, potassium hydroxide or barium hydroxide, or alkali metal carbonates or alkaline earth metal carbonates, such as sodium carbonate, potassium carbonate or calcium carbonate. Particular preference is given to sodium hydroxide or lithium hydroxide.

Acids suitable for the ester cleavage are, in general, sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, if appropriate with added water. Preference is given to hydrogen chloride or trifluoroacetic acid in the case of the tert-butyl esters and to hydrochloric acid in the case of the methyl esters.

The ester cleavage is generally carried out in a temperature range of from 0° C. to +100° C., preferably at from +0° C. to +50° C. The nitrile hydrolysis is generally carried out in a temperature range of from +50° C. to +150° C., preferably at from +80° C. to +120° C.

Reactions mentioned can be carried out at atmospheric, elevated or reduced pressure (for example from 0.5 to 5 bar). In general, reactions are carried out at atmospheric pressure.

The compounds of the formula (I) according to the invention in which Z represents a group of the formula

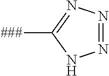

can be prepared by reacting compounds of the formula (IV) in which Z¹ represents cyano in an inert solvent with an alkali metal azide in the presence of ammonium chloride or with trimethylsilyl azide, if appropriate in the presence of a catalyst.

Inert solvents for this reaction are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, or other solvents, such as dimethyl sulfoxide, dimethylformamide, N,N′-dimethylpropyleneurea (DMPU) or N-methylpyrrolidone (NMP). It is also possible to use mixtures of the solvents mentioned. Preference is given to using toluene.

A suitable azide reagent is in particular sodium azide in the presence of ammonium chloride or trimethylsilyl azide. The latter reaction can advantageously be carried out in the presence of a catalyst. Suitable for this purpose are in particular compounds such as di-n-butyltin oxide, trimethylaluminum or zinc bromide. Preference is given to using trimethylsilyl azide in combination with di-n-butyltin oxide.

The reaction is generally carried out in a temperature range of from +50° C. to +150° C., preferably at from +60° C. to +110° C. The reaction can be carried out at atmospheric, elevated or reduced pressure (for example from 0.5 to 5 bar). In general, the reaction is carried out at atmospheric pressure.

The compounds of the formula (I) according to the invention in which Z represents a group of the formula

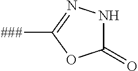

can be prepared by converting compounds of the formula (IV) in which Z¹ represents methoxycarbonyl or ethoxycarbonyl [y=0] initially in an inert solvent with hydrazine into compounds of the formula (VII)

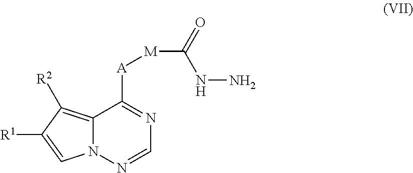

in which A, M, R¹ and R² each have the meanings given above,
and then reacting these in an inert solvent with phosgene or a phosgene equivalent, such as, for example, N,N′-carbonyldiimidazole.

Suitable inert solvents for the first step of this reaction sequence are in particular alcohols, such as methanol, ethanol, n-propanol, isopropanol, n-butanol or tert-butanol, or ethers, such as diethyl ether, dioxane, tetrahydrofuran, glycol dimethyl ether or diethylene glycol dimethyl ether. It is also possible to use mixtures of these solvents. Preference is given to using a mixture of methanol and tetrahydrofuran. The second reaction step is preferably carried out in an ether, in particular in tetrahydrofuran. The reactions are generally carried out in a temperature range of from 0° C. to +70° C., under atmospheric pressure.

The compounds of the formula (I) according to the invention in which L¹ represents a group of the formula *-L^{1A}-V-L^{1B}-** in which L^{1A}, L^{1B} and V have the meanings given above can alternatively also be prepared by converting compounds of the formula (VIII)

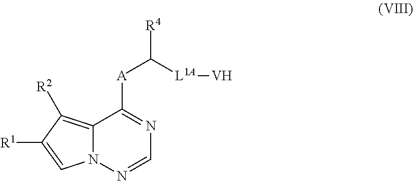

in which A, L^{1A}, V, R¹, R² and R⁴ each have the meanings given above
in the presence of a base, if appropriate in an inert solvent, with a compound of the formula (IX)

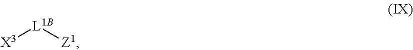

in which L^{1B} and Z¹ have the meanings given above
and
X³ represents a leaving group, such as, for example, halogen, mesylate, tosylate or triflate,
or, in the case that L^{1B} represents —CH₂CH₂— with a compound of the formula (X)

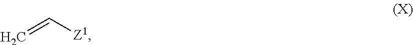

in which Z¹ has the meaning given above,
into compounds of the formula (IV-A)

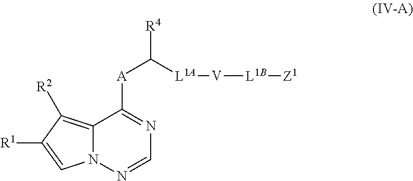

in which A, L^{1A}, L^{1B}, V, Z¹, R¹, R² and R⁴ each have the meanings given above,
and then reacting these further, in a manner corresponding to the process described above.

The compounds of the formula (VIII) can—analogously to the preparation of the compounds (IV)—be obtained by base-catalyzed reaction of a compound of the formula (II) or (V) with a compound of the formula (XI) or (XII)

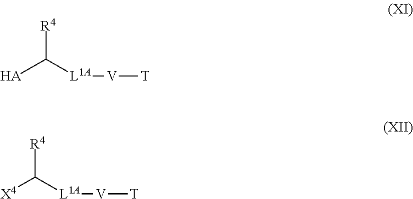

in which A, L^{1A}, V and R⁴ each have the meanings given above,
T represents hydrogen or a temporary O- or N-protective group
and
X⁴ represents a leaving group, such as, for example, halogen, mesylate, tosylate or triflate,
(cf. also Reaction Schemes 1 and 2 below).

In an analogous manner, the compounds of the formula (I) according to the invention in which L³ represents a group of the formula -W—CH₂- or -W—CH₂—CH₂- in which W has the meanings given above can also be prepared by converting compounds of the formula (XIII)

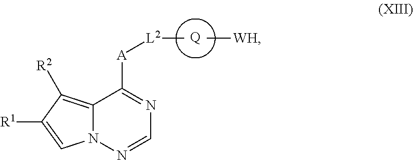

in which A, L², Q, W, R¹ and R² each have the meanings given above,
in the presence of a base, if appropriate in an inert solvent, with a compound of the formula (XIV)

X⁵—(CH₂)Z¹ (XIV),

in which Z¹ has the meaning given above,
n represents the number 1 or 2
and
X⁵ represents a leaving group, such as, for example, halogen, mesylate, tosylate or triflate,
or in the case that L³ represents -W—CH₂CH₂- with a compound of the formula (X)

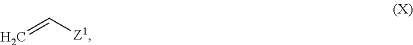

in which Z¹ has the meaning given above,
into compounds of the formula (IV-B)

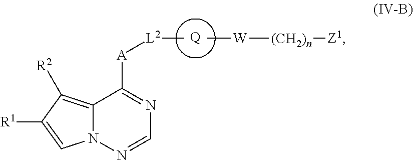

in which A, L², Q, W, Z¹, R¹, R² and n each have the meanings given above,
and then reacting these further according to one of the processes described above.

The compounds of the formula (XIII) can—analogously to the preparation of the compounds (IV)—be obtained by base-catalyzed reaction of a compound of the formula (II) or (V) with a compound of the formula (XV) or (XVI)

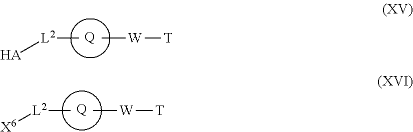

in which A, L², Q and W each have the meanings given above,
T represents hydrogen or a temporary O- or N-protective group
and
X⁶ represents a leaving group, such as, for example, halogen, mesylate, tosylate or triflate,
(cf. also Reaction Schemes 1 and 2 below).

For the process steps (VIII)+(IX) and (X)→(IV-A), (II)+(XI)→(VIII), (V)+(XII)→(VIII), (XIII)+(XIV) and (X)→(IV-B), (II)+(XV)→(XIII) and (V)+(XVI)→(XIII), the reaction parameters described above for the reactions (II)+(III)→(IV) and (V)+(VI) →(IV), such as solvents, bases and reaction temperatures, are used in an analogous manner.

The compounds of the formulae (II) and (V) can be prepared by aminating 2-cyano-pyrroles of the formula (XVII)

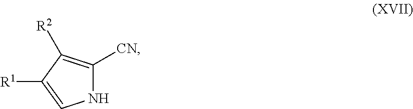

in which R¹ and R² have the meanings given above,
with the aid of a hydroxylamine derivative such as o-mesitylenesulfonylhydroxyl-amine or (aminooxy)[bis(4-methoxyphenyl)]phosphine oxide [cf. Smulik et al., Organic Letters 2003, 5 (22), 4187] in the presence of a base to give compounds of the formula (XVIII)

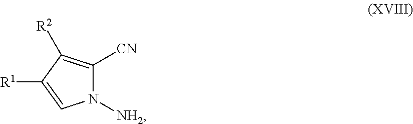

in which R¹ and R² have the meanings given above,
and then either
[a] condensing these compounds with formamide to give compounds of the formula (V-A)

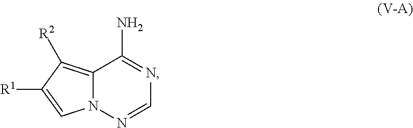

in which R¹ and R² have the meanings given above,
and, if appropriate, then reacting them with isoamyl nitrite in the presence of a chloride source such as hydrogen chloride or copper(II) chloride to give compounds of the formula (II-A)

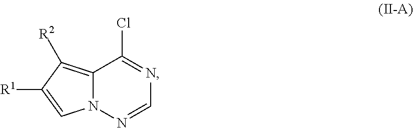

in which R¹ and R² have the meanings given above,
or
[b] condensing these compounds in the presence of acetic anhydride with formic acid to give compounds of the formula (V-B)

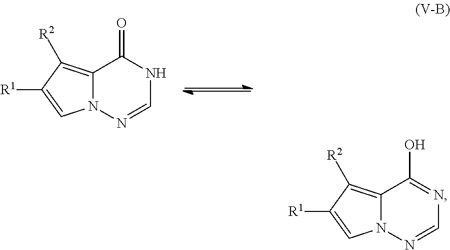

in which R¹ and R² have the meanings given above,
and, if appropriate, then converting them with the aid of phosphor oxychloride into compounds of the formula (II-A) (see also Reaction Scheme 3 below).

The compounds of the formula (XVII) for their part can be prepared, for example, by reacting compounds of the formula (XIX)

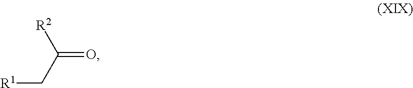

in which R¹ and R² have the meanings given above,
with a dimethylformamide acetal to give compounds of the formula (XX)

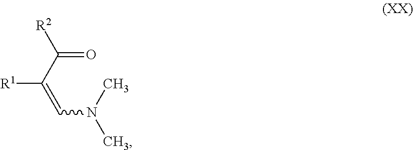

in which R¹ and R² have the meanings given above,
and then condensing these under acidic conditions with 2-amino-2-cyanoacetamide (see also Reaction Scheme 3 below).

The compounds of the formulae (III), (VI), (IX), (X), (XI), (XII), (XIV), (XV), (XVI) and (XIX) are commercially available, known from the literature or can be prepared analogously to processes known from the literature.

The preparation of the compounds according to the invention can be illustrated by way of example by the synthesis schemes below:

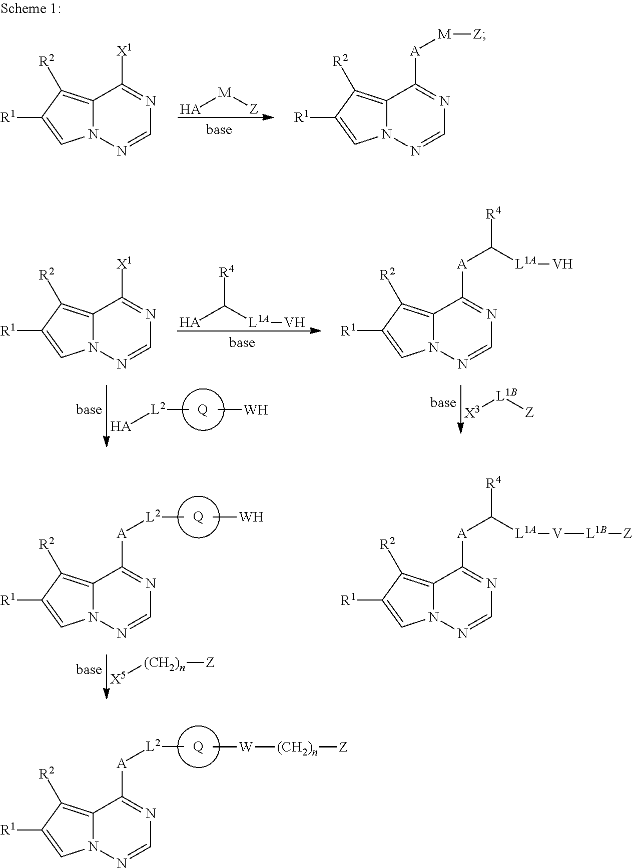

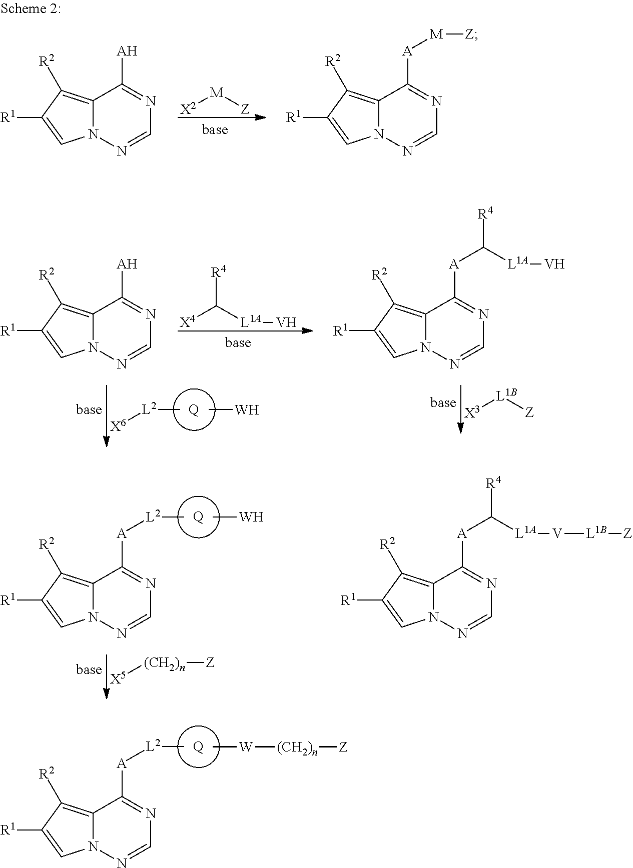

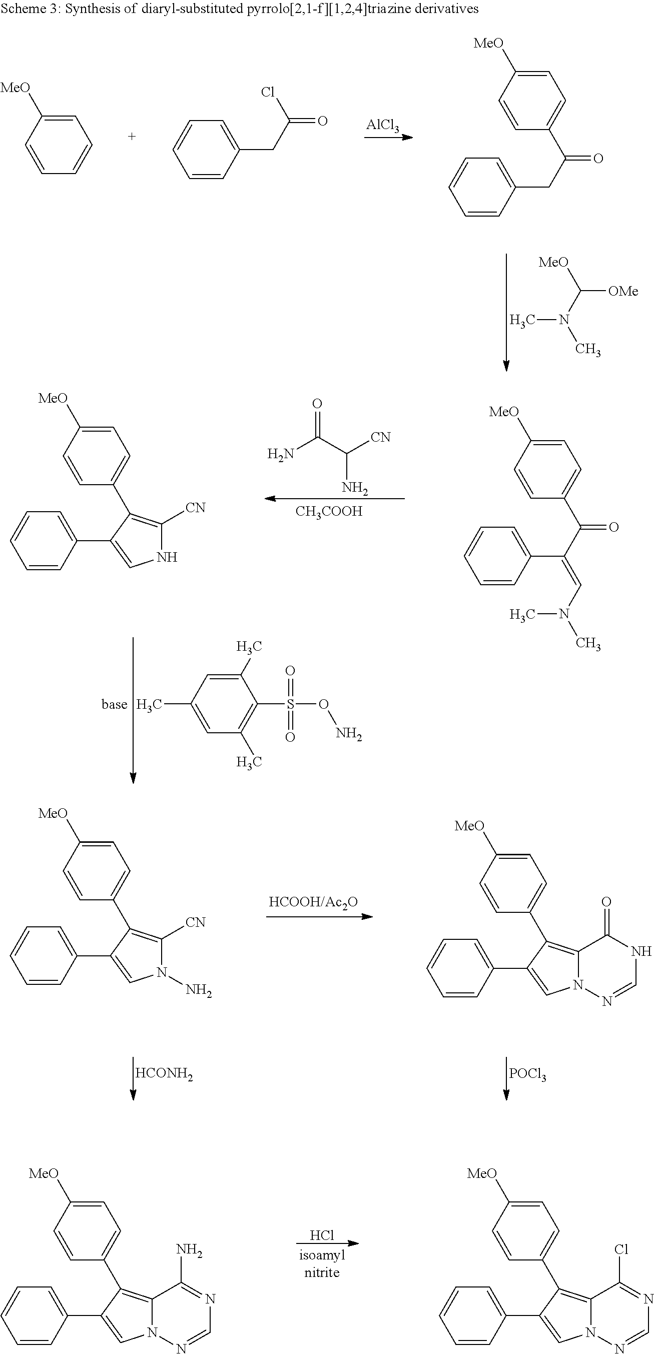

The compounds according to the invention possess valuable pharmacological properties and can be used for the prevention and treatment of diseases in humans and animals. The compounds according to the invention are chemically and metabolically stable, non-prostanoid activators of the IP receptor which mimic the biological action of PGI₂.

They are thus suitable in particular for the prophylaxis and/or treatment of cardiovascular diseases such as stable and unstable angina pectoris, of hypertension and heart failure, pulmonary hypertension, for the prophylaxis and/or treatment of thromboembolic diseases and ischaemias such as myocardial infarction, stroke, transient and ischaemic attacks and subarachnoid haemorrhage, and for the prevention of restenosis such as after thrombolytic treatments, percutaneous transluminal angioplasty (PTA), coronary angioplasty (PTCA) and bypass surgery.

The compounds according to the invention are particularly suitable for the treatment and/or prophylaxis of pulmonary hypertension (PH) including its various manifestations. The compounds of the invention are therefore particularly suitable for the treatment and/or prophylaxis of pulmonary arterial hypertension (PAH) and its subtypes such as idiopathic and familial pulmonary arterial hypertension, and the pulmonary arterial hypertension which is associated for example with portal hypertension, fibrotic disorders, HIV infection or inappropriate medications or toxins. The compounds of the invention can also be used for the treatment and/or prophylaxis of other types of pulmonary hypertension. Thus, for example, they can be employed for the treatment and/or prophylaxis of pulmonary hypertension associated with left atrial or left ventricular disorders and with left heart valve disorders. In addition, the compounds of the invention are suitable for the treatment and/or prophylaxis of pulmonary hypertension associated with chronic obstructive pulmonary disease, interstitial pulmonary disease, pulmonary fibrosis, sleep apnoea syndrome, disorders with alveolar hypoventilation, altitude sickness and pulmonary development impairments.

The compounds of the invention are furthermore suitable for the treatment and/or prophylaxis of pulmonary hypertension based on chronic thrombotic and/or embolic disorders such as, for example, thromboembolism of the proximal pulmonary arteries, obstruction of the distal pulmonary arteries and pulmonary embolism. The compounds of the invention can further be used for the treatment and/or prophylaxis of pulmonary hypertension connected with sarcoidosis, histiocytosis X or lymphangioleiomyomatosis, and where the pulmonary hypertension is caused by external compression of vessels (lymph nodes, tumor, fibrosing mediastinitis).

In addition, the compounds according to the invention can also be used for the treatment and/or prophylaxis of peripheral and cardial vascular diseases, peripheral occlusive diseases (PAOD, PVD) and disturbances of peripheral blood flow. Furthermore, the compounds according to the invention can be used for the treatment of arteriosclerosis, hepatitis, asthmatic diseases, chronic obstructive pulmonary diseases (COPD), pulmonary edema, fibrosing lung diseases such as idiopathic pulmonary fibrosis (IPF) and ARDS, inflammatory vascular diseases such as scleroderma and lupus erythematosus, renal failure, arthritis and osteoporosis, and also for the prophylaxis and/or treatment of cancers, especially of metastasizing tumors.

Moreover, the compounds according to the invention can also be used as an addition to the preserving medium of an organ transplant, e.g. kidneys, lungs, heart or islet cells.

The present invention further relates to the use of the compounds according to the invention for the treatment and/or prophylaxis of diseases, and especially of the aforementioned diseases.

The present invention further relates to the use of the compounds according to the invention for the production of a medicinal product for the treatment and/or prophylaxis of diseases, and especially of the aforementioned diseases.

The present invention further relates to a method for the treatment and/or prophylaxis of diseases, especially of the aforementioned diseases, using an effective amount of at least one of the compounds according to the invention.

The compounds of the invention can be employed alone or, if required, in combination with other active ingredients. The present invention further relates to medicaments comprising at least one of the compounds of the invention and one or more further active ingredients, especially for the treatment and/or prophylaxis of the aforementioned disorders. Suitable active ingredients for combinations are by way of example and preferably:

organic nitrates and NO donors such as, for example, sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), such as, for example, inhibitors of phosphodiesterases (PDE) 1, 2, 3, 4 and/or 5, especially PDE 5 inhibitors such as sildenafil, vardenafil and tadalafil;
NO-independent but heme-dependent stimulators of guanylate cyclase such as in particular the compounds described in WO 00/06568, WO 00/06569, WO 02/42301 and WO 03/095451;
NO- and heme-independent activators of guanylate cyclase, such as in particular the compounds described in WO 01/19355, WO 01/19776, WO 01/19778, WO 01/19780, WO 02/070462 and WO 02/070510;
compounds which inhibit human neutrophile elastase (HNE), such as, for example, sivelestat, DX-890 (Reltran), elafin or in particular the compounds described in WO 03/053930, WO 2004/020410, WO 2004/020412, WO 2004/024700, WO 2004/024701, WO 2005/080372, WO 2005/082863 and WO 2005/082864;
compounds which inhibit the signal transduction cascade, for example and preferably from the group of kinase inhibitors, in particular from the group of tyrosine kinase and/or serine/threonine kinase inhibitors;
compounds which inhibit soluble epoxide hydrolase (sEH), such as, for example, N,N′-dicyclohexylurea, 12-(3-adamantan-1-ylureido)dodecanoic acid or 1-adamantan-1-yl-3-{5-[2-(2-ethoxyethoxy)ethoxy]pentyl}urea;
compounds which influence the energy metabolism of the heart, such as by way of example and preferably etomoxir, dichloroacetate, ranolazine or trimetazidine;
agonists of VPAC receptors, such as by way of example and preferably the vasoactive intestinal polypeptide (VIP);
agents having an antithrombotic effect, for example and preferably from the group of platelet aggregation inhibitors, of anticoagulants or of profibrinolytic substances;
active ingredients which lower blood pressure, for example and preferably from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors and diurectics; and/or active ingredients which alter lipid metabolism, for example and preferably from the group of thyroid receptor agonists, cholesterol synthesis inhibitors such as by way of example and preferably HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

In a preferred embodiment of the invention, the compounds of the invention are administered in combination with a kinase inhibitor such as by way of example and preferably canertinib, imatinib, gefitinib, erlotinib, lapatinib, lestaurtinib, lonafarnib, pegaptinib, pelitinib, semaxanib, tandutinib, tipifarnib, vatalanib, sorafenib, sunitinib, bortezomib, lonidamine, leflunomide, fasudil or Y-27632.

Agents having an antithrombotic effect preferably mean compounds from the group of platelet aggregation inhibitors, of anticoagulants or of profibrinolytic substances.

In a preferred embodiment of the invention, the compounds of the invention are administered in combination with a platelet aggregation inhibitor such as by way of example and preferably aspirin, clopidogrel, ticlopidine or dipyridamole.

In a preferred embodiment of the invention, the compounds of the invention are administered in combination with a thrombin inhibitor such as by way of example and preferably ximelagatran, melagatran, bivalirudin or clexane.

In a preferred embodiment of the invention, the compounds of the invention are administered in combination with a GPIIb/IIIa antagonist such as by way of example and preferably tirofiban or abciximab.

In a preferred embodiment of the invention, the compounds of the invention are administered in combination with a factor Xa inhibitor such as by way of example and preferably rivaroxaban, DU-176b, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or a low molecular weight (LMW) heparin derivative.

In a preferred embodiment of the invention, the compounds of the invention are administered in combination with a vitamin K antagonist such as by way of example and preferably coumarin.

Agents which lower blood pressure preferably mean compounds from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors, and diuretics.

In a preferred embodiment of the invention, the compounds of the invention are administered in combination with a calcium antagonist such as by way of example and preferably nifedipine, amlodipine, verapamil or diltiazem.

In a preferred embodiment of the invention, the compounds of the invention are administered in combination with an alpha-1 receptor blocker such as by way of example and preferably prazosin.

In a preferred embodiment of the invention, the compounds of the invention are administered in combination with a beta-receptor blocker such as by way of example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin All antagonist such as by way of example and preferably losartan, candesartan, valsartan, telmisartan or embusartan. In a preferred embodiment of the invention, the compounds of the invention are administered in combination with an ACE inhibitor such as by way of example and preferably enalapril, captopril, lisinopril, ramipril, delapril, fosinopril, quinopril, perindopril or trandopril.

In a preferred embodiment of the invention, the compounds of the invention are administered in combination with an endothelin antagonist such as by way of example and preferably bosentan, darusentan, ambrisentan or sitaxsentan.

In a preferred embodiment of the invention, the compounds of the invention are administered in combination with a renin inhibitor such as by way of example and preferably aliskiren, SPP-600 or SPP-800.

In a preferred embodiment of the invention, the compounds of the invention are administered in combination with a mineralocorticoid receptor antagonist such as by way of example and preferably spironolactone or eplerenone.

In a preferred embodiment of the invention, the compounds of the invention are administered in combination with a Rho kinase inhibitor such as by way of example and preferably fasudil, Y-27632, SLx-2119, BF-66851, BF-66852, BF-66853, KI-23095, SB-772077, GSK-269962A or BA-1049.

In a preferred embodiment of the invention, the compounds of the invention are administered in combination with a diuretic such as by way of example and preferably furosemide.

Agents which alter lipid metabolism preferably mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and lipoprotein(a) antagonists.

In a preferred embodiment of the invention, the compounds of the invention are administered in combination with a CETP inhibitor such as by way of example and preferably torcetrapib (CP-529 414), JJT-705 or CETP vaccine (Avant).

In a preferred embodiment of the invention, the compounds of the invention are administered in combination with a thyroid receptor agonist such as by way of example and preferably D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins such as by way of example and preferably lovastatin, simvastatin, pravastatin, fluvastatin, atorvastatin, rosuvastatin, cerivastatin or pitavastatin.

In a preferred embodiment of the invention, the compounds of the invention are administered in combination with a squalene synthesis inhibitor such as by way of example and preferably BMS-188494 or TAK-475.

In a preferred embodiment of the invention, the compounds of the invention are administered in combination with an ACAT inhibitor such as by way of example and preferably avasimibe, melinamide, pactimibe, eflucimibe or SMP-797.

In a preferred embodiment of the invention, the compounds of the invention are administered in combination with an MTP inhibitor such as by way of example and preferably implitapide, BMS-201038, R-103757 or JTT-130.

In a preferred embodiment of the invention, the compounds of the invention are administered in combination with a PPAR-gamma agonist such as by way of example and preferably pioglitazone or rosiglitazone.

In a preferred embodiment of the invention, the compounds of the invention are administered in combination with a PPAR-delta agonist such as by way of example and preferably GW-501516 or BAY 68-5042.

In a preferred embodiment of the invention, the compounds of the invention are administered in combination with a cholesterol absorption inhibitor such as by way of example and preferably ezetimibe, tiqueside or pamaqueside.

In a preferred embodiment of the invention, the compounds of the invention are administered in combination with a lipase inhibitor such as by way of example and preferably orlistat.

In a preferred embodiment of the invention, the compounds of the invention are administered in combination with a polymeric bile acid adsorbent such as by way of example and preferably cholestyramine, colestipol, colesolvam, CholestaGel or colestimide.

In a preferred embodiment of the invention, the compounds of the invention are administered in combination with a bile acid reabsorption inhibitor such as by way of example and preferably ASBT (═IBAT) inhibitors such as, for example, AZD-7806, S-8921, AK-105, BAR1—1741, SC-435 or SC-635.

In a preferred embodiment of the invention, the compounds of the invention are administered in combination with a lipoprotein(a) antagonist such as by way of example and preferably gemcabene calcium (CI-1027) or nicotinic acid.

The present invention further relates to medicaments comprising at least one of the compounds according to the invention, usually in combination with one or more inert, non-toxic, pharmaceutically suitable excipients, and their use for the purposes mentioned above.

The compounds of the invention may have systemic and/or local effects. For this purpose, they can be administered in a suitable way such as, for example, by the oral, parenteral, pulmonary, nasal, sublingual, lingual, buccal, rectal, dermal, transdermal, conjunctival or otic route or as implant or stent.

The compounds of the invention can be administered in administration forms suitable for these administration routes.

Suitable for oral administration are administration forms which function according to the prior art and deliver the compounds of the invention rapidly and/or in a modified manner, and which contain the compounds of the invention in crystalline and/or amorphized and/or dissolved form, such as, for example, tablets (uncoated and coated tablets, for example having coatings which are resistant to gastric juice or are insoluble or dissolve with a delay and control the release of the compound of the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates or sterile powders.

Suitable for the other routes of administration are, for example, pharmaceutical forms for inhalation (inter alia powder inhalers, nebulizers), nasal drops, solutions or sprays, tablets for lingual, sublingual or buccal administration, films/wafers or capsules, suppositories, preparations for the ears and eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

Oral or parenteral administration are preferred, especially oral and intravenous administration.

The compounds of the invention can be converted into the stated administration forms. This can take place in a manner known per se by mixing with inert, non-toxic, pharmaceutically suitable excipients. These excipients include inter alia carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorings (e.g. inorganic pigments such as, for example, iron oxides) and masking flavors and/or odors.

It has generally proved to be advantageous on parenteral administration to administer amounts of about 0.001 to 1 mg/kg, preferably about 0.01 to 0.5 mg/kg of body weight to achieve effective results. On oral administration, the dosage is about 0.01 to 100 mg/kg, preferably about 0.01 to 20 mg/kg, and very particularly preferably 0.1 to 10 mg/kg of body weight.

It may nevertheless be necessary where appropriate to deviate from the stated amounts, in particular as a function of body weight, administration route, individual response to the active ingredient, type of preparation and time or interval over which administration takes place. Thus, in some cases it may be sufficient to make do with less than the aforementioned minimum amount, whereas in other cases the upper limit mentioned must be exceeded. Where relatively large amounts are administered, it may be advisable to distribute these in a plurality of single doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data of liquid/liquid solutions are based in each case on the volume.

### A. EXAMPLES

### Abbreviations

abs. absolute
Ac acetyl
Ac₂O acetic anhydride
aq. aqueous, aqueous solution
c concentration
TLC thin-layer chromatography
DCI direct chemical ionization (in MS)
DIBAH diisobutylaluminum hydride

### DMF N,N-dimethylformamide

DMSO dimethyl sulfoxide
ee enantiomeric excess
EI electron impact ionization (in MS)
eq equivalent(s)
ESI electrospray ionization (in MS)
m.p. melting point
sat. saturated
h hour(s)
HPLC high pressure liquid chromatography
cat. catalytic
conc. concentrated
LC-MS liquid chromatography-coupled mass spectrometry
Me methyl
min minute(s)
MS mass spectrometry
NMR nuclear magnetic resonance spectrometry
rac. racemic
RP reversed phase (in HPLC)
RT room temperature
Rₜ retention time (in HPLC)
TFA trifluoroacetic acid
THF tetrahydrofuran

### LC-MS Methods:

### Method 1:

MS instrument type: Micromass ZQ; HPLC instrument type: HP 1100 Series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 2:

Instrument: Micromass QuattroPremier with Waters HPLC Acquity; column: Thermo Hypersil GOLD 1.9μ, 50 mm×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A; oven: 50° C.; flow rate: 0.33 ml/min; UV detection: 210 nm.

### Method 3:

MS instrument type: Micromass ZQ; HPLC instrument type: Waters Alliance 2795; column: Phenomenex Synergi 2.5 μMAX-RP 100A Mercury, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A →0.1 min 90% A→3.0 min 5% A→4.0 min 5% A→4.01 min 90% A; flow rate: 2 ml/min; oven: 50° C.; UV detection: 210 nm.

### Method 4:

Instrument: Micromass Quattro Micro MS with HPLC Agilent Series 1100; column: Thermo Hypersil GOLD 3μ, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A→4.01 min 100% A (flow rate 2.5 ml/min)→5.00 min 100% A; oven: 50° C.; flow rate: 2 ml/min; UV detection: 210 nm.

### Starting Materials and Intermediates

### Example 1A

### tert-Butyl (2E,6R)-6-hydroxyhept-2-enoate

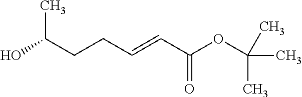

Solution A: 10.71 g (267.7 mmol) of 60% sodium hydride are suspended in 150 ml of abs. THF, and 43.3 ml (276.7 mmol) of tert-butyl P,P-dimethylphosphonoacetate are added dropwise with cooling. The mixture is stirred at RT, and after about 30 min a solution is formed.

187.4 ml (187.4 mmol) of a 1 M solution of DIBAH in THF are added dropwise to a solution, cooled to −78° C., of 17.87 g (178.5 mmol) of (R)-γ-valerolactone [(5R)-5-methyldihydrofuran-2(3H)-one] in 200 ml of abs. THF. The solution is stirred at −78° C. for 1 h, and solution A, prepared above, is then added. After the end of the addition, the mixture is slowly warmed to RT and stirred at RT overnight. The reaction mixture is added to 300 ml of ethyl acetate and extracted by stirring with 50 ml of concentrated potassium sodium tartrate solution. After phase separation, the aqueous phase is re-extracted with ethyl acetate. The organic phases are combined, washed with sat. sodium chloride solution, dried over magnesium sulfate and concentrated under reduced pressure. The residue is purified by chromatography on silica gel (mobile phase: cyclohexane/ethyl acetate 5:1). This gives 32.2 g (90.1% of theory) of the target product which contains small amounts of the cis-isomer.

MS (DCI): m/z=218 (M+NH₄)⁺

¹H-NMR (400 MHz, DMSO-d₆): δ=6.70 (dt, 1H), 5.73 (d, 1H), 4.44 (d, 1H), 3.58 (m, 1H), 2.28-2.13 (m, 2H), 1.47-1.40 (m, 2H), 1.45 (s, 9H), 1.04 (d, 3H).

### Example 2A

### tert-Butyl(−)-6-hydroxyheptanoate

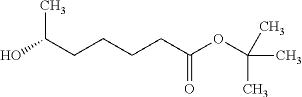

32.2 g (160.8 mmol) of tert-butyl (2E,6R)-6-hydroxyhept-2-enoate are dissolved in 200 ml of ethanol, and 1.7 g of 10% palladium on carbon are added. The mixture is stirred at RT under an atmosphere of hydrogen (atmospheric pressure) for 2 h and then filtered off through Celite. The filtrate is concentrated under reduced pressure. The residue gives, after chromatography on silica gel (mobile phase: cyclohexane/ethyl acetate 10:1→6:1), 15.66 g of the target product (48.1% of theory).

MS (DCI): m/z=220 (M+NH₄)⁺

¹H-NMR (400 MHz, CDCl₃): δ=3.85-3.75 (m, 1H), 2.22 (t, 2H), 1.68-1.54 (m, 2H), 1.53-1.30 (m, 4H), 1.45 (s, 9H), 1.18 (d, 3H).

[α]_{D}²⁰=−21°, c=0.118, chloroform.

### Example 3A

### 1-(4-Methoxyphenyl)-2-phenylethanone

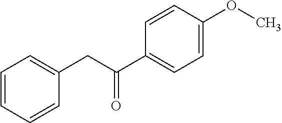

At 0° C., 10.0 g (64.7 mmol) of phenylacetyl chloride were added dropwise to a suspension of 9.86 g (73.9 mmol) of aluminum trichloride in 200 ml of 1,2-dichloroethane. The mixture was stirred at 0° C. for 5 min, and 6.66 g (61.6 mmol) of anisole were then added dropwise (internal temperature 5-8° C.). After the addition had ended, cooling was removed and the mixture was stirred at RT for 2.5 h. With vigorous stirring, the mixture was then added to ice-water. After addition of conc. hydrochloric acid, the mixture was extracted with 1,2-dichloroethane. The organic phase was washed successively with water, 1 N aqueous sodium hydroxide solution and sat. sodium chloride solution, dried over sodium sulfate and concentrated under reduced pressure. The residue was dried under high vacuum. This gave 16.24 g of the target product, which was used without further purification for the subsequent reaction.

LC-MS (Method 1): Rₜ=2.50 min; m/z=227 (M+H)⁺

¹H-NMR (400 MHz, DMSO-d₆): δ=8.04 (d, 2H), 7.35-7.20 (m, 5H), 7.05 (d, 2H), 4.32 (s, 2H), 3.86 (s, 3H).

### Example 4A

### 3-(Dimethylamino)-1-(4-methoxyphenyl)-2-phenylprop-2-en-1-one

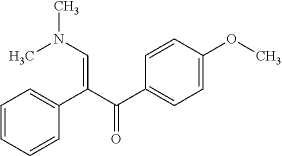

21.7 g (95.9 mmol) of 1-(4-methoxyphenyl)-2-phenylethanone were dissolved in 110 ml of toluene and warmed to 50° C., and 19.1 ml (143.9 mmol) of N,N-dimethylformamide dimethyl acetal were added. The reaction mixture was stirred at 80° C. overnight and then, after cooling, concentrated under reduced pressure. The residue was repeatedly taken up in toluene and in each case concentrated to dryness under reduced pressure again. The solid obtained was triturated with petroleum ether, filtered off and dried under high vacuum. This gave 24.26 g of the target product (89.9% of theory).

¹H-NMR (400 MHz, DMSO-d₆): δ=7.37 (d, 2H), 7.31-7.18 (m, 4H), 7.10 (d, 2H), 6.87 (d, 2H), 3.28 (s, 3H), 2.68 (s, 6H).

### Example 5A

### 3-(4-Methoxyphenyl)-4-phenyl-1H-pyrrole-2-carbonitrile

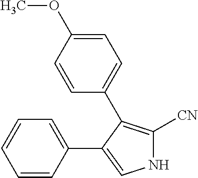

8.45 g (85.3 mmol) of 2-amino-2-cyanoacetamide were added to a solution of 24.0 g (85.3 mmol) of 3-(dimethylamino)-1-(4-methoxyphenyl)-2-phenylprop-2-en-1-one in 144 ml of glacial acetic acid. The reaction mixture was heated at 80° C. for 1 h. After cooling to RT, 5 ml of conc. sulfuric acid were added and the reaction mixture was stirred at RT for a further hour. The mixture was then added to water and extracted with dichloromethane. The organic phase was washed with water, sat. sodium bicarbonate solution and sat. sodium chloride solution, dried over sodium sulfate and concentrated under reduced pressure. The crude product was purified by chromato-graphy on silica gel (mobile phase: cyclohexane/ethyl acetate 6:1 →4:1). This gave 13.49 g of the target product (49.2% of theory).

LC-MS (Method 1): Rₜ=2.65 min; m/z=275 (M+H)⁺

¹H-NMR (400 MHz, DMSO-d₆): δ=12.53 (s, 1H), 7.41 (s, 1H), 7.29-7.15 (m, 7H), 6.94 (d, 2H), 3.80 (s, 3H).

### Example 6A

### 1-Amino-3-(4-methoxyphenyl)-4-phenyl-1H-pyrrole-2-carbonitrile

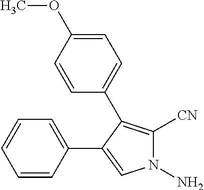

Under an atmosphere of argon, a solution of 960 mg (2.5 mmol) of 3-(4-methoxyphenyl)-4-phenyl-1H-pyrrole-2-carbonitrile in 3.4 ml of DMF was added dropwise to an ice-cooled suspension of 700 mg (17.5 mmol, 60%) of sodium hydride in 30 ml of DMF. The mixture was stirred at 0° C. for 10 min. A solution of 1130 mg (about 5 mmol) of o-mesitylenesulfonylhydroxylamine (still slightly water-moist, freshly prepared according to Synthesis, 1972, 140; careful: may explode when dry!) in about 5 ml of DMF was then added. The reaction mixture was stirred at 0° C. for 3 h and then diluted with ethyl acetate, and sat. ammonium chloride solution was added carefully. The organic phase was washed with sat. sodium chloride solution, dried over sodium sulfate and concentrated under reduced pressure. The residue was purified by chromatography on silica gel (mobile phase: cyclohexane/ethyl acetate 7:1). This gave 594 mg of the target product (58.7% of theory).

LC-MS (Method 2): Rₜ=1.23 min; m/z=290 (M+H)⁺

¹H-NMR (400 MHz, DMSO-d₆): δ=7.31 (s, 1H), 7.29-7.11 (m, 7H), 6.95 (d, 2H), 6.49 (s, 2H), 3.79 (s, 3H).

### Example 7A

### 4-Amino-5-(4-methoxyphenyl)-6-phenylpyrrolo[2,1-f][1,2,4]triazine

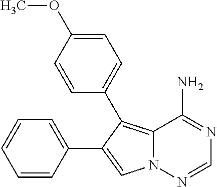

590 mg (2.04 mmol) of 1-amino-3-(4-methoxyphenyl)-4-phenyl-1H-pyrrole-2-carbonitrile were initially charged in 1.6 ml of formamide and heated in a microwave at 140° C. After 10 h, the mixture was cooled to RT, and water and a lot of ethyl acetate were added. The separated organic phase was substantially concentrated under reduced pressure and the suspension that remained was treated with aceto-nitrile. The precipitated solid was filtered off with suction and dried under high vacuum. This gave 456 mg of the target product (70.7% of theory).

LC-MS (Method 3): Rₜ=1.77 min; m/z=317 (M+H)⁺

¹H-NMR (400 MHz, DMSO-d₆): δ=8.03 (s, 1H), 7.89 (s, 1H), 7.30-7.16 (m, 7H), 7.03 (d, 2H), 4.95 (br. s, 2H), 3.81 (s, 3H).

### Example 8A

### 5-(4-Methoxyphenyl)-6-phenylpyrrolo[2,1-f][1,2,4]triazin-4(3H)-one

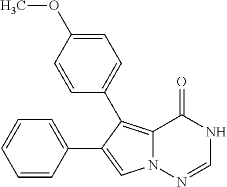

2.4 ml (25.9 mmol) of acetic anhydride were cooled to 0° C., and 1.2 ml (31.1 mmol) of formic acid were added a little at a time. The mixture was stirred at 0° C. for 30 min. 300 mg (1.04 mmol) of 1-amino-3-(4-methoxyphenyl)-4-phenyl-1H-pyrrole-2-carbonitrile were then added. The reaction mixture was heated to 130° C. (bath temperature) and stirred for 24 h. After cooling, the reaction mixture was concentrated under high vacuum and the residue was taken up in a little DMSO. The product was isolated by preparative RP-HPLC (mobile phase: acetonitrile/water gra-dient). This gave 47.8 mg of the target compound (14.5% of theory).

LC-MS (Method 1): Rₜ=2.30 min; m/z=318 (M+H)⁺

¹H-NMR (500 MHz, DMSO-d₆): δ=11.54 (s, 1H), 7.84 (s, 1H), 7.83 (s, 1H), 7.25 (m, 2H), 7.21-7.15 (m, 4H), 6.85 (d, 2H), 3.78 (s, 3H).

### Example 9A

### 4-Chloro-5-(4-methoxyphenyl)-6-phenylpyrrolo[2,1-f][1,2,4]triazine

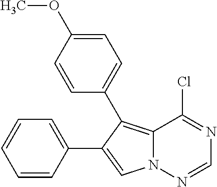

At RT, 0.45 ml (4.79 mmol) of phosphorus oxychloride was added to 76 mg (0.24 mmol) of 5-(4-methoxyphenyl)-6-phenylpyrrolo[2,1-f][1,2,4]triazin-4(3H)-one. The suspension was heated under reflux for 3 h, during which time the solid dissolved. After cooling, the mixture was diluted with dichloromethane, and water and ammonia solution were added (pH of the aqueous phase about 9). After phase separation, the aqueous phase was reextracted twice with dichloromethane. All organic phases were combined, dried over magnesium sulfate and concentrated under reduced pressure. Preparative RP-HPLC (mobile phase: acetonitrile/water gradient) gave 62.7 mg of the target product (78.0% of theory).

LC-MS (Method 3): Rₜ=2.43 min; m/z=336 (M+H)⁺

¹H-NMR (500 MHz, DMSO-d₆): δ=8.61 (s, 1H), 8.42 (s, 1H), 7.30-7.23 (m, 7H), 6.97 (d, 2H), 3.80 (s, 3H).

### Working Examples

### Example 1

### Ethyl (6-{[5-(4-methoxyphenyl)-6-phenylpyrrolo[2,1-f][1,2,4]triazin-4-yl]amino}hexanoate

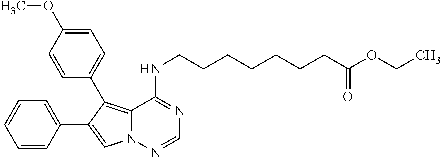

At 0° C., 11.6 mg (0.289 mmol, 60%) of sodium hydride were added to a mixture of 87.0 mg (0.275 mmol) of 4-amino-5-(4-methoxyphenyl)-6-phenylpyrrolo[2,1-f][1,2,4]triazine and 92.0 mg (0.412 mmol) of ethyl 6-bromohexanoate in 0.32 ml of abs. DMF. The reaction mixture was slowly warmed to RT and stirred at this temperature for 2 h and then added to water. The mixture was extracted thoroughly with ethyl acetate and the organic phase was washed with sat. sodium chloride solution, dried over magnesium sulfate and concentrated under reduced pressure. The crude product was purified by preparative RP-HPLC (mobile phase: acetonitrile/water gradient). This gave 49.4 mg of the target compound (39.2% of theory).

LC-MS (Method 3): Rₜ=2.53 min; m/z=459 (M+H)⁺

¹H-NMR (500 MHz, DMSO-d₆): δ=7.99 (s, 1H), 7.94 (s, 1H), 7.29 (d, 2H), 7.28-7.17 (m, 5H), 7.03 (d, 2H), 5.28 (t, 1H), 4.03 (q, 2H), 3.82 (s, 3H), 3.34 (q, 2H), 2.24 (t, 2H), 1.49-1.41 (m, 2H), 1.40-1.43 (m, 2H), 1.17 (t, 3H), 1.13-1.05 (m, 2H).

### Example 2

### tert-Butyl (6R)-6-{[5-(4-methoxyphenyl)-6-phenylpyrrolo[2,1-f][1,2,4]triazin-4-yl]oxy}heptanoate

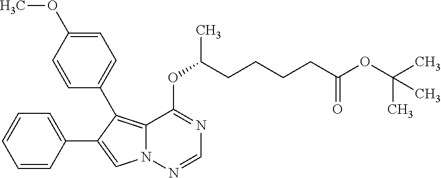

At −5° C. and under an atmosphere of argon, 0.24 ml (0.24 mmol) of a 1 M solution of P4-phosphazene base in THF was added dropwise to a solution of 61.0 mg (0.182 mmol) of 4-chloro-5-(4-methoxyphenyl)-6-phenylpyrrolo[2,1-f][1,2,4]triazine and 51.4 mg (0.254 mmol) of tert-butyl(−)-6-hydroxyheptanoate in 0.27 ml of abs. THF. After the addition had ended, the mixture was warmed to 0° C. and stirred for 40 min. The reaction mixture was then concentrated under reduced pressure and the residue was purified directly by preparative RP-HPLC (mobile phase: acetonitrile/water gradient). This gave 21.2 mg of the target product (23.3% of theory).

LC-MS (Method 4): Rₜ=3.29 min; m/z=502 (M+H)⁺

¹H-NMR (400 MHz, CDCl₃): δ=7.99 (s, 1H), 7.81 (s, 1H), 7.30-7.20 (m, 7H), 6.83 (d, 2H), 5.30 (m, 1H), 3.84 (s, 3H), 2.12 (t, 2H), 1.54-1.45 (m, 4H), 1.42 (s, 9H), 1.28 (d, 3H), 1.22-1.10 (m, 2H).

### General Procedure A: Hydrolysis of Methyl or Ethyl Esters to the Corresponding Carboxylic Acids

At RT, 1.5 to 10 eq. of sodium hydroxide, as a 1 N aqueous solution, are added to a solution of the methyl or ethyl ester in THF or THF/methanol (1:1) (concentration about 0.05 to 0.5 mol/l). The mixture is stirred at RT for a period of 0.5-18 h and then neutralized or acidified slightly with 1 N hydrochloric acid. If a solid precipitates out, the product can be isolated by filtration, washing with water and drying under high vacuum. Alternatively, the target compound is isolated directly from the crude product, if appropriate after extractive work-up with dichloromethane, by preparative RP-HPLC (mobile phase: acetonitrile/water gradient) or purified by trituration with an inert solvent.

The Working Example below was obtained according to General Procedure A:

| Example | Structure | Analytical data |
|---|---|---|
| 3 | 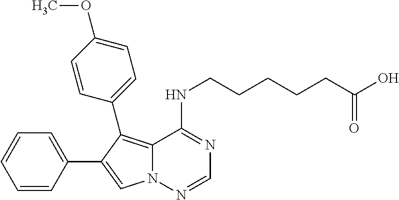 | LC-MS (Method 1): Rₜ = 2.49 min; m/z = 431 (M + H)⁺ ¹H-NMR (400 MHz, DMSO-d₆): δ = 12.02 (s, 1H), 8.01 (s, 1H), 7.95 (s, 1H), 7.31 (d, 2H), 7.28- 7.16 (m, 5H), 7.05 (d, 2H), 5.29 (t, 1H), 3.84 (s, 3H), 3.40-3.35 (m, 2H), 2.17 (t, 2H), 1.47-1.32 (m, 4H), 1.14-1.05 (m, 2H). |

### General Procedure B: Cleavage of Tert-Butyl Esters to the Corresponding Carboxylic Acids

At from 0° C. to RT, trifluoroacetic acid (TFA) is added dropwise to a solution of the tert-butyl ester in dichloromethane (concentration from 0.1 to 1.0 mol/l; additionally, if required, one drop of water), until a dichloromethane/TFA ratio of about 2:1 to 1:2 is reached. The mixture is stirred at RT for 1-18 h and then concentrated under reduced pressure. Alternatively, the reaction mixture is diluted with dichloromethane, washed with water and sat. sodium chloride solution, dried and concentrated under reduced pressure. The crude product can, if required, be purified for instance by preparative RP-HPLC (mobile phase: acetonitrile/water gradient).

The Working Example below was obtained according to General Procedure B:

| Example | Structure | Analytical data |
|---|---|---|
| 4 | 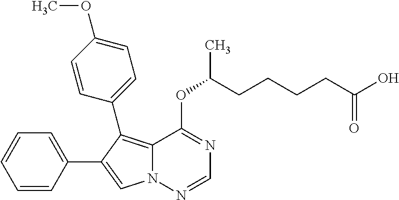 | LC-MS (Method 4): Rₜ = 2.66 min; m/z = 446 (M + H)⁺ ¹H-NMR (500 MHz, DMSO-d₆): δ = 11.94 (br. s, 1H), 8.20 (s, 1H), 8.14 (s, 1H), 7.29-7.14 (m, 7H), 6.89 (d, 2H), 5.27 (m, 1H), 3.78 (s, 3H), 2.10 (t, 2H), 1.49- 1.32 (m, 4H), 1.21 (d, 3H), 1.15- 1.02 (m, 2H). [α]_{D}²⁰ = −81°, c = 0.105, chloro- form. |

### B. ASSESSMENT OF PHARMACOLOGICAL EFFICACY

The pharmacological action of the compounds according to the invention can be demonstrated in the following assays:

### B-1. Studies of Binding to Prostacyclin Receptors (IP Receptors) of Human Thrombocyte Membranes

Thrombocyte membranes are obtained by centrifuging 50 ml of human blood (Buffy coats with CDP Stabilizer, from Maco Pharma, Langen) for 20 min at 160×g. Remove the supernatant (platelet-rich plasma, PRP) and then centrifuge again at 2000×g for 10 min at room temperature. Resuspend the sediment in 50 mM tris(hydroxymethyl)aminomethane, which has been adjusted to a pH of 7.4 with 1 N hydrochloric acid, and store at −20° C. overnight. On the next day, centrifuge the suspension at 80 000×g and 4° C. for 30 min. Discard the supernatant. Resuspend the sediment in 50 mM tris(hydroxymethyl)aminomethane/hydrochloric acid, 0.25 mM ethylene diamine tetraacetic acid (EDTA), pH 7.4, and then centrifuge once again at 80 000×g and 4° C. for 30 min. Take up the membrane sediment in binding buffer (50 mM tris(hydroxymethyl)-aminomethane/hydrochloric acid, 5 mM magnesium chloride, pH 7.4) and store at −70° C. until the binding test.

For the binding test, incubate 3 nM ³H-Iloprost (592 GBq/mmol, from AmershamBioscience) for 60 min with 300-1000 μg/ml of human thrombocyte membranes per charge (max. 0.2 ml) in the presence of the test substances at room temperature. After stopping, add cold binding buffer to the membranes and wash with 0.1% bovine serum albumin. After adding Ultima Gold Scintillator, quantify the radioactivity bound to the membranes using a scintillation counter. The nonspecific binding is defined as radioactivity in the presence of 1 μM Iloprost (from Cayman Chemical, Ann Arbor) and is as a rule <25% of the bound total radioactivity. The binding data (IC₅₀ values) are determined using the program GraphPad Prism Version 3.02.

Representative results for the compounds according to the invention are shown in Table 1:

TABLE 1

| Example No. | IC₅₀ [nM] |
|---|---|
| 3 | 636 |
| 4 | 224 |

### B-2. IP-Receptor Stimulation on Whole Cells

The IP-agonistic action of test substances is determined by means of the human erythroleukaemia cell line (HEL), which expresses the IP-receptor endogenously [Murray, R., FEBS Letters 1989, 1: 172-174]. For this, the suspension cells (4×10⁷ cells/ml) are incubated with the particular test substance for 5 minutes at 30° C. in buffer [10 mM HEPES (4-(2-hydroxyethyl)-1-piperazineethanesulfonic acid)/PBS (phosphate-buffered saline, from Oxoid, UK)], 1 mM calcium chloride, 1 mM magnesium chloride, 1 mM IBMX (3-isobutyl-1-methylxanthine), pH 7.4. Next, the reaction is stopped by addition of 4° C. cold ethanol and the charges are stored for a further 30 minutes at 4° C. Then the samples are centrifuged at 10 000×g and 4° C. The resultant supernatant is discarded and the sediment is used for determination of the concentration of cyclic adenosine monophosphate (cAMP) in a commercially available cAMP-radioimmunoassay (from IBL, Hamburg). In this test, IP agonists lead to an increase in cAMP concentration, but IP antagonists have no effect. The effective concentration (EC₅₀ value) is determined using the program GraphPad Prism Version 3.02.

### B-3. Inhibition of Thrombocyte Aggregation In Vitro

Inhibition of thrombocyte aggregation is determined using blood from healthy test subjects of both sexes. Mix 9 parts blood with one part 3.8% sodium citrate solution as coagulant. Centrifuge the blood at 900 rev/min for 20 min. Adjust the pH value of the platelet-rich plasma obtained to pH 6.5 with ACD solution (sodium citrate/citric acid/glucose). Then remove the thrombocytes by centrifugation, take up in buffer and centrifuge again. Take up the thrombocyte deposit in buffer and additionally resuspend with 2 mmol/l calcium chloride.

For the measurements of aggregation, incubate aliquots of the thrombocyte suspension with the test substance for 10 min at 37° C. Next, aggregation is induced by adding ADP and is determined by the turbidometric method according to Born in the aggregometer at 37° C. [Born G.V.R., J. Physiol. (London) 168, 178-179 (1963)].

### B-4. Measurement of Blood Pressure of Anaesthetized Rats

Anaesthetize male Wistar rats with a body weight of 300-350 g with thiopental (100 mg/kg i.p.). After tracheotomy, catheterize the arteria femoralis for blood pressure measurement. Administer the test substances as solution, orally by oesophageal tube or intravenously via the femoral vein in a suitable vehicle.

### B-5. PAH Model in the Anaesthetized Dog

In this animal model of pulmonary arterial hypertension (PAH), mongrel dogs having a body weight of about 25 kg are used. Narcosis is induced by slow i.v. administration of 25 mg/kg of sodium thiopental (Trapanal®) and 0.15 mg/kg of alcuronium chloride (Alloferin®) and maintained during the experiment by continuous infusion of 0.04 mg/kg/h of Fentanyl®, 0.25 mg/kg/h of droperidol (Dehydro-benzperidol®) and 15 μg/kg/h of alcuronium chloride (Alloferin®). Reflectory effects on the pulse by lowering of the blood pressure are kept to a minimum by autonomous blockage [continuous infusion of atropin (about 10 μg/kg/h) and propranolol (about 20 μg/kg/h)]. After intubation, the animals are ventilated using a ventilator with constant tidal volume such that an end-tidal CO₂ concentration of about 5% is reached. Ventilation takes place with ambient air enriched with about 30% oxygen (normoxa). For measuring the hemodynamic parameters, a liquid-filled catheter is implanted into the femoral artery for measuring the blood pressure. A double-lumiger Swan-Ganz® catheter is introduced via the jugular vein into the pulmonary artery (distal lumen for measuring the pulmonary arterial pressure, proximal lumen for measuring the central venous pressure). The left-ventricular pressure is measured following introduction of a micro-tip catheter (Millar® Instruments) via the carotid artery into the left ventricle, and from this, the dP/dt value is derived as a measure for the contractility. Substances are administered i.v. via the femoral vein. The hemodynamic signals are recorded and evaluated using pressure sensors/amplifiers and PONEMAH® as data acquisition software.

To induce acute pulmonary hypertension, the stimulus used is either hypoxia or continuous infusion of thromboxan A₂ or a thromboxan A₂ analog. Acute hypoxia is induced by gradually reducing the oxygen in the ventilation air to about 14%, such that the mPAP increases to values of >25 mm Hg. If the stimulus used is a thromboxan A₂ analog, 0.21-0.32 μg/kg/min of U-46619 [9,11-dideoxy-9α,11α-epoxymethanoprostaglandin F_{2α} (from Sigma)] are infused to increase the mPAP to >25 mm Hg.

### B-6. PAH Model in Anaesthetized Gottingen Minipig

In this animal model of pulmonary arterial hypertension (PAH), Gottingen Minipigs

having a body weight of about 25 kg are used. Narcosis is induced by 30 mg/kg of ketamine (Ketavet®) i.m., followed by i.v. administration of 10 mg/kg of sodium thiopental (Trapanal®); during the experiment, it is maintained by inhalation narcosis using enfluran (2-2.5%) in a mixture of ambient air enriched with about 30-35% oxygen/N₂O (1:1.5). For measuring the hemodynamic parameters, a liquid-filled catheter is implanted into the carotid artery for measuring the blood pressure. A double-lumiger Swan-Ganz® catheter is introduced via the jugular vein into the pulmonary artery (distal lumen for measuring the pulmonary arterial pressure, proximal lumen for measuring the central venous pressure). The left-ventricular pressure is measured following introduction of a micro-tip catheter (Millar® Instruments) via the carotid artery into the left ventricle, and from this, the dP/dt value is derived as a measure for the contractility. Substances are administered i.v. via the femoral vein. The hemodynamic signals are recorded and evaluated using pressure sensors/amplifiers and PONEMAH® as data acquisition software.

To induce acute pulmonary hypertension, the stimulus used is continuous infusion of a thromboxan A₂ analog. Here, 0.12-0.14 μg/kg/min of U-46619 [9,11-dideoxy-9α,11α-epoxymethanoprostaglandin F_{2α} (from Sigma)] are infused to increase the mPAP to >25 mm Hg.

### C. EXEMPLARY EMBODIMENTS OF PHARMACEUTICAL COMPOSITIONS

The compounds of the invention can be converted into pharmaceutical preparations in the following ways:

### Tablet:

### Composition:

100 mg of the compound of the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

### Production:

The mixture of compound of the invention, lactose and starch is granulated with a 5% strength solution (m/m) of the PVP in water. The granules are mixed with the magnesium stearate for 5 minutes after drying. This mixture is compressed with a conventional tablet press (see above for format of the tablet). A guideline compressive force for the compression is 15 kN.

Suspension which can be Administered Orally:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol, and the compound of the invention is added to the suspension. The water is added while stirring. The mixture is stirred for about 6 h until the swelling of the Rhodigel is complete.

Solution which can be Administered Orally:

### Composition:

500 mg of the compound of the invention, 2.5 g of polysorbate and 97 g of polyethylene glycol 400.20 g of oral solution correspond to a single dose of 100 mg of the compound according to the invention.

### Production:

The compound of the invention is suspended in the mixture of polyethylene glycol and polysorbate with stirring. The stirring process is continued until the compound according to the invention has completely dissolved.

i.v. Solution:

The compound of the invention is dissolved in a concentration below the saturation solubility in a physiologically tolerated solvent (e.g. isotonic saline solution, 5% glucose solution and/or 30% PEG 400 solution). The solution is sterilized by filtration and used to fill sterile and pyrogen-free injection containers.

1. A compound of the formula (I) 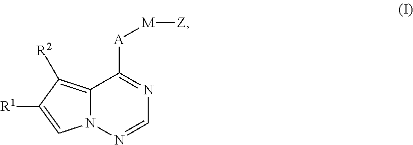
in which
A represents O or N—R³ in which
R³ represents hydrogen, (C₁-C₆)-alkyl, (C₃-C₇)-cycloalkyl or (C₄-C₇)-cycloalkenyl,
M represents a group of the formula 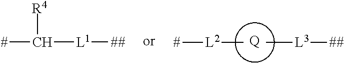
in which
# represents the point of attachment to the group A
and
## represents the point of attachment to the group Z,
R⁴ represents hydrogen or (C₁-C₄)-alkyl which may be substituted by hydroxyl or amino,
L¹ represents (C₁-C₇)-alkanediyl or (C₂-C₇)-alkenediyl which may be mono- or disubstituted by fluorine, or a group of the formula *-L^{1A}-V-L^{1B}-** in which
* represents the point of attachment to the group —CHR⁴,
** represents the point of attachment to the group Z,
L^{1A} represents (C₁-C₅)-alkanediyl which may be mono- or disubstituted by identical or different radicals from the group consisting of (C₁-C₄)-alkyl and (C₁-C₄)-alkoxy,
L^{1B} represents a bond or (C₁-C₃)-alkanediyl which may be mono- or disubstituted by fluorine,
and
V represents O or N—R⁵ in which
R⁵ represents hydrogen, (C₁-C₆)-alkyl or (C₃-C₇)-cycloalkyl,
L² represents a bond or (C₁-C₄)-alkanediyl,
L³ represents (C₁-C₄)-alkanediyl which may be mono- or disubstituted by fluorine and in which a methylene group may be replaced by O or N—R⁶ in which
R⁶ represents hydrogen, (C₁-C₆)-alkyl or (C₃-C₇)-cycloalkyl,
or represents (C₂-C₄)-alkenediyl,
and
Q represents (C₃-C₇)-cycloalkyl, (C₄-C₇)-cycloalkenyl, phenyl, 5- to 7-membered hetero-cyclyl or 5- or 6-membered heteroaryl, each of which may be substituted up to two times by identical or different radicals from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino,
where (C₁-C₄)-alkyl for its part may be substituted by hydroxyl, (C₁-C₄)-alkoxy, amino, mono- or di-(C₁-C₄)-alkylamino,
Z represents a group of the formula 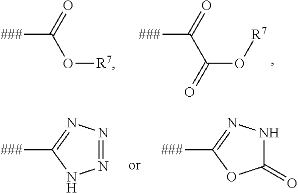
in which
### represents the point of attachment to the group L¹ or L³
and
R⁷ represents hydrogen or (C₁-C₄)-alkyl,
and
R¹ and R² are identical or different and independently of one another represent (C₃-C₇)-cyclo-alkyl, (C₄-C₇)-cycloalkenyl, phenyl, 5- to 7-membered heterocyclyl or 5- or 6-membered hetero-aryl, each of which may be mono- to trisubstituted by identical or different radicals from the group consisting of halogen, cyano, nitro, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-acyl, amino, mono-(C₁-C₆)-alkylamino, di-(C₁-C₆)-alkylamino and (C₁-C₆)-acylamino,
where (C₁-C₆)-alkyl and (C₁-C₆)-alkoxy for their part may in each case be substituted by cyano, hydroxyl, (C₁-C₄)-alkoxy, (C₁-C₄)-alkylthio, amino, mono- or di-(C₁-C₄)-alkylamino,
or
R¹ and/or R² represent(s) phenyl in which two radicals attached to adjacent ring carbon atoms together form a group of the formula —O—CH₂—O—, —O—CHF—O—, —O—CF₂—O—, —O—CH₂—CH₂—O— or —O—CF₂—CF₂—O—,
or one of its salts.

2. The compound of the formula (I) as claimed in claim 1 in which
A represents O or NH,
M represents a group of the formula 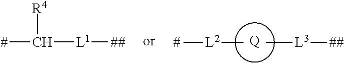
in which
# represents the point of attachment to the group A
and
## represents the point of attachment to the group Z,
R⁴ represents hydrogen, methyl or ethyl,
L¹ represents (C₃-C₇)-alkanediyl, (C₃-C₇)-alkenediyl or a group of the formula *-L^{1A}-V-L^{1B}-** in which
* represents the point of attachment to the group —CHR⁴,
** represents the point of attachment to the group Z,
L^{1A} represents (C₁-C₃)-alkanediyl which may be mono- or disubstituted by methyl,
L^{1B} represents (C₁-C₃)-alkanediyl
and
V represents O or N—CH₃,
L² represents a bond, methylene, ethane-1,1-diyl or ethane-1,2-diyl,
L³ represents (C₁-C₃)-alkanediyl or a group of the formula -W—CH₂- or -W—CH₂—CH₂- in which
 represents the point of attachment to the ring Q,
 represents the point of attachment to the group Z
and
W represents O or N—R⁶ in which
R⁶ represents hydrogen or (C₁-C₃)-alkyl,
and
Q represents cyclobutyl, cyclopentyl, cyclopentenyl, cyclohexyl, pyrrolidinyl, piperidinyl, tetrahydrofuranyl, tetrahydropyranyl, morpholinyl or phenyl, each of which may be substituted up to two times by identical or different radicals from the group consisting of fluorine, methyl, ethyl, trifluoromethyl, hydroxyl, methoxy and ethoxy,
Z represents a group of the formula 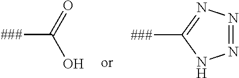
in which
### represents the point of attachment to the group L¹ or L³,
and
R¹ and R² are identical or different and independently of one another represent cyclopenten-1-yl, cyclohexen-1-yl, phenyl, thienyl or pyridyl, each of which may be mono- or disubstituted by identical or different radicals from the group consisting of fluorine, chlorine, cyano, (C₁-C₄)-alkyl, (C₂-C₄)-alkenyl, (C₁-C₄)-alkoxy, trifluoromethyl and trifluoromethoxy.

3. The compound of the formula (I) as claimed in claim 1 in which
A represents O or NH,
M represents the group of the formula 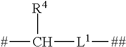
in which
# represents the point of attachment to the group A
and
## represents the point of attachment to the group Z,
R⁴ represents hydrogen or methyl,
and
L¹ represents butane-1,4-diyl, pentane-1,5-diyl or a group of the formula *-L^{1A}-O-L^{1B}-** in which
* represents the point of attachment to the group —CHR⁴,
** represents the point of attachment to the group Z,
L^{1A} represents methylene or ethane-1,2-diyl which may be mono- or disubstituted by methyl,
and
L^{1B} represents methylene or ethane-1,2-diyl,
Z represents the group of the formula 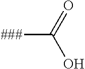
in which
### represents the point of attachment to the group L¹,
R¹ represents phenyl which may be substituted by fluorine or chlorine,
and
R² represents phenyl which may be substituted by methyl, ethyl, methoxy or ethoxy.

4. A process for preparing compounds as defined in claim 1 in which Z represents —COOH or —C(═O)—COOH, characterized in that either
[A] compounds of the formula (II) 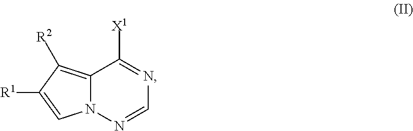
in which R¹ and R² have the meanings given in claim 1
and
X¹ represents a leaving group such as, for example, halogen, in particular chlorine, are reacted in an inert solvent in the presence of a base with a compound of the formula (III) 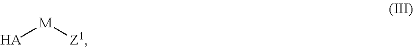
in which A and M have the meanings given in claim 1
and
Z¹ represents cyano or a group of the formula —[C(O)]_{y}—COOR^{7A} in which
y represents the number 0 or 1
and
R^{7A} represents (C₁-C₄)-alkyl,
to give compounds of the formula (IV) 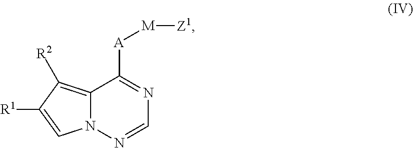
in which A, M, Z¹, R¹ and R² each have the meanings given above,
or
[B] compounds of the formula (V) 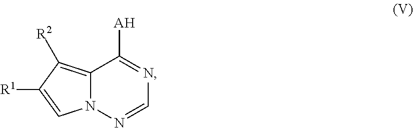
in which A, R¹ and R² each have the meanings given in claim 1, are reacted in an inert solvent in the presence of a base with a compound of the formula (VI) 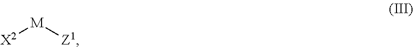
in which M has the meaning given in claim 1 and Z¹ has the meaning given above
and
X² represents a leaving group such as, for example, halogen, mesylate, tosylate or triflate, to give compounds of the formula (IV) 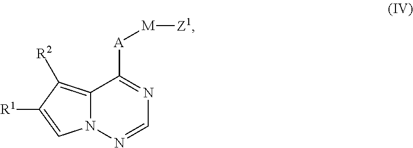
in which A, M, Z¹, R¹ and R² each have the meanings given above,
and the compounds of the formula (IV) are then converted by hydrolysis of the ester or cyano group Z¹ into the carboxylic acids of the formula (I-A) 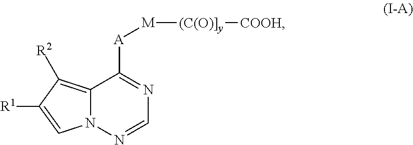
in which A, M, R¹, R² and y each have the meanings given above,
and these are, if appropriate, converted into their solvates, salts and/or solvates of the salts using the appropriate (i) solvents and/or (ii) bases or acids.

5-6. (canceled)

7. A medicament comprising a compound as defined in claim 1 in combination with an inert non-toxic pharmaceutically acceptable auxiliary.

8-9. (canceled)

10. A method for the treatment and/or prophylaxis of angina pectoris, pulmonary hypertension, thromboembolic disorders and peripheral occlusive diseases in humans and animals comprising the step of administering to a mammal an effective amount of at least one compound as defined in claim 1.
